(19) United States
(12) Patent Application Publication
(10) Pub. No.: US 2025/0228909 A1
(43) Pub. Date: Jul. 17, 2025

(54) THERAPEUTIC COMPOSITIONS AND METHODS

(71) Applicant: Carus Animal Health Limited, Reigate (GB)

(72) Inventors: Gregory FIRTH, Sheffield (GB); Rajesh Muru ODEDRA, Stevenage (GB)

(21) Appl. No.: 19/022,757

(22) Filed: Jan. 15, 2025

(30) Foreign Application Priority Data
Jan. 16, 2024 (GB) 2400580.3

(51) Int. Cl. A61K 35/76; C12N 7/00

(52) U.S. Cl. CPC A61K 35/76; C12N 7/00; C12N 2795/00032

(57) ABSTRACT

The present disclosure relates to the reduction of methane production in humans, in particular humans suffering from irritable bowel syndrome. The disclosure provides viruses capable of inhibiting the formation of methane in vivo in the gastrointestinal tract of a human. The disclosure also provides compositions, including pharmaceutical compositions, comprising a virus capable of reducing the formation of methane in vivo in the human gastrointestinal tract. The disclosure further provides methods of reducing the production of methane in humans, comprising administering to the human a virus capable of inhibiting the production of methane in vivo in the gastrointestinal tract.

### CROSS-REFERENCE TO RELATED APPLICATIONS

This application claims priority to UK Patent Application No. 2400580.3, filed Jan. 16, 2024, the entire contents of which are incorporated by reference herein.

### SEQUENCE LISTING

The instant application contains a Sequence Listing which has been submitted electronically in XML format and is hereby incorporated by reference in its entirety. The XML copy, created on Jan. 23, 2025, is named 16408.0003-SL.xml and is 40,750 bytes in size.

### FIELD

The present disclosure relates to the reduction of methane production in humans, in particular humans suffering from irritable bowel syndrome. In particular, the disclosure provides viruses capable of inhibiting the formation of methane in vivo in the gastrointestinal tract of a human. The disclosure also provides compositions, including pharmaceutical compositions, comprising a virus capable of reducing the formation of methane in vivo in the human gastrointestinal tract. The disclosure further provides methods of reducing the production of methane in humans, comprising administering to the human a virus capable of inhibiting the production of methane in vivo in the gastrointestinal tract.

### BACKGROUND

Irritable bowel syndrome (IBS) is a functional gastrointestinal disorder affecting the human alimentary tract with a worldwide prevalence estimated at 12%. The disease affects the female population approximately twice as much as the male population and is most common in people younger than 45 years of age.

Bowel movements vary considerably in their nature among IBS patients and this gives rise to categorization into three main subtypes: diarrhea-predominant IBS (D-IBS), constipation-predominant IBS (C-IBS), and mixed IBS (M-IBS). Faecal microbial composition differs among IBS patients depending on their stool quality and IBS categorization. Stool firmness strongly correlates with faecal microflora diversity, there being a multispecies richness in people with firm stools which decreases across the spectrum of stool consistency reaching a minimum for diarrhea afflicted individuals.

Methanogenic archaea (commonly referred to as methanogens) are the predominant biological sources of methane in nature and Methanobrevibacter smithii is the predominant methanogen in the human intestine. These methanogens may contribute 10% of the gut microbiome and are found in the colon but also can be demonstrated to reside in the small bowel, especially when small intestine bacterial overgrowth exists. An increased abundance of these methanogens is found in harder stools of C-IBS patients compared with diarrhea of D-IBS patients and elevated production of methane is associated with constipation. Indeed, methane is a putative neurotransmitter acting on the cholinergic pathway of the enteric nervous system causing delayed intestinal transit time of ingesta and promoting segmental (non-propagating) peristaltic contractions.

IBS is an important health care concern as it greatly affects patients' quality of life. In particular, C-IBS may negatively impact general wellbeing through abdominal pain, bloating, and altered bowel habits. Given that C-IBS patients experience increased methane production due to an increased abundance of methanogens, a reduction of methanogenesis in these patients is one therapeutic strategy to achieve symptomatic relief. Previously, this has been performed using pharmaceuticals such as the prodrug lactone form of lovastatin, or by antibiotics such as rifaximin and neomycin. However, these drugs have drawbacks in that they disrupt the enteric microbiome through indiscriminate effects. Thus, there is a need for more targeted strategies for reducing the production of methane in patients suffering from IBS, particularly those with C-IBS, because elevated production of methane is associated with adverse symptoms including discomfort due to bloating, and constipation, so a reduction in methane production in these patients would improve their quality of life. The present disclosure addresses this need.

PCT/EP2023/069722, U.S. Ser. No. 18/352,760, U.S. Ser. No. 18/492,384, UK 20230010863, and AR 2023P101846 disclose solid and semi-solid compositions for administration to a ruminant which comprise a substance capable of inhibiting the production of methane in vivo in the ruminant, such as a virus capable of inhibiting archaea that produce methane in vivo in a ruminant. The present disclosure builds on that disclosure to address an unmet need in humans.

### SUMMARY

In a first aspect, there is provided a virus capable of inhibiting the formation of methane in vivo in the gastrointestinal tract of a human for use in a method of reducing the production of methane in a human subject, the method comprising administering the virus to the subject.

In a second aspect, there is provided a composition, for example a pharmaceutical composition, comprising a virus capable of inhibiting the formation of methane in vivo in the human gastrointestinal tract. The pharmaceutical according to this aspect may be for use in a method of reducing the production of methane in a human subject, the method comprising administering the composition to the subject.

In a third aspect, there is provided a method of reducing the production of methane in a human subject, comprising administering to the subject a virus capable of inhibiting the formation of methane in vivo in the gastrointestinal tract.

The viruses, compositions, and methods provide an effective means to reduce methane production in humans, particularly in patients with C-IBS who exhibit elevated levels of methane production. The reduction of methane production in these patients reduces symptoms associated with C-IBS, including constipation.

### Definitions

The term “comprising,” “including,” and “includes” as used herein means that at least all of the listed elements must be present, but other elements that are not mentioned may also be present.

The term “consisting of” as used herein means that only the listed elements must be present, such that other elements that are not mentioned are not present.

The terms “constipation-predominant irritable bowel syndrome” and “C-IBS” as used interchangeably herein define the subtype of irritable bowel syndrome which is defined as abdominal pain with hard or lumpy stools characterizing more than 25% of bowel movements and loose or watery stools constituting less than 25% of bowel movements for an average of three days per month over a span of last three months using the Rome IV criteria for irritable bowel syndrome.

The term “virus” as used herein includes double-stranded or single-stranded RNA or DNA viruses, which infect cells of bacteria archaea, plants and/or animals.

The term “substrate” as used herein is understood to mean any solid phase material to which a virus may be immobilized.

The term “virus capable of inhibiting the production of methane” is used herein to refer to a virus capable of inhibiting the production of methane in vivo in a human.

As used herein, the term “about,” as applied to one or more values of interest, refers to a value that is similar to a stated reference value. In some embodiments, the term “about” refers to a range of values that fall within 25% of the reference value in either direction (greater than or less than the reference value). In some embodiments, the term “about” refers to values that fall within 20%, 19%, 18%, 17%, 16%, 15%, 14%, 13%, 12%, 11%, 10%, 9%, 8%, 7%, 6%, 5%, 4%, 3%, 2%, 1%, or less of the stated reference value (except where such number would exceed 100% of a possible value).

The term “coated” as used herein with respect to a particular composition refers to the presence of a coating on at least 10% of the external surface of the composition. In some embodiments, the coating is present on at least 20%, 30%, 40%, 50%, 60%, 70%, 80%, 90%, or 95% of the external surface.

The term “encapsulated” as used herein with respect to coating of compositions means that the composition is enclosed within the coating (in other words, the composition is contained entirely within the coating).

As used herein, “location-controlled release” means that the virus is released a particular location within the human digestive tract after it is administered.

As used herein, “dietary supplement” refers to a composition that is added to a subject's regular food intake. Dietary supplements may include the viruses described herein alone, or alongside other ingredients intended to supplement the diet, such as vitamins and minerals, fibre, herbs and other botanical extracts including flower remedies, homeopathic remedies, amino acids, enzymes and live microbials, probiotics, prebiotics, or any combination thereof.

Dietary supplements may be formulated in a wide variety of ways including as oils, gummies, drops, capsules, rapid-melt formulations, lozenges, oral sprays, chewing gums, gels, powders, premixed drinks, meal replacement shakes, or bars.

As used herein, “stabilization agent” refers to a substance capable of retaining the desired properties of a virus in a composition comprising the virus and the stabilization agent. In some embodiments, the stabilization agent is a substrate, and the virus is immobilized onto the substrate. In some embodiments, the stabilization agent is a sugar (e.g., sucrose, sorbitol, trehalose, saccharose, mannitol, lactose, etc.), a preservative, an antioxidant, a dispersant (e.g., polyvinylpyrrolidone 40, dextran, PEG, etc.), a thermal stability agent (e.g., urea), a buffer (e.g., Tris, PBS, sodium phosphate, acetate, borate, HEPES, MOPS), a polymer (e.g., PEG), an amino acid, or combinations thereof. As used herein, a “buffer” may comprise water, but a “buffer” cannot consist of water.

### DETAILED DESCRIPTION

The present disclosure relates to a virus capable of inhibiting the formation of methane in vivo in the gastrointestinal tract of a human, for use in a method of reducing the production of methane in a human.

The present disclosure further relates to a composition, such as a pharmaceutical composition, comprising a virus capable of reducing the formation of methane in vivo in the human gastrointestinal tract. The composition may be in the form of a liquid, syrup, capsule, tablet, bead, granule, pellet, lozenge, suppository, powder, or pill. In some embodiments, the composition may be in the form of tablet which is coated with an enteric coating. The composition may be for use in a method of reducing the production of methane in a human subject, the method comprising administering the composition to the subject.

The present disclosure also relates to methods for reducing methane production in a human subject, comprising administering to the subject a virus capable of inhibiting the production of methane in vivo in the gastrointestinal tract.

In some embodiments, the human has IBS. In some embodiments, the human has C-IBS.

In some embodiments, the virus capable of inhibiting the production of methane in vivo in the gastrointestinal tract is administered orally or rectally.

The virus capable of inhibiting the production of methane in vivo in the gastrointestinal tract may be a virus which interrupts the metabolic pathway of methane production in the gastrointestinal tract, and/or may be a virus capable of disrupting the enteric microbiome. In some embodiments, the virus is an inhibitor of methanogens. In some embodiments, the virus is an inhibitor of methanogens in the Methanobrevibacter genus. In some embodiments, the virus is an inhibitor of Methanobrevibacter smithii.

In some embodiments, the virus capable of inhibiting methane production in vivo in a human is a stabilized virus. In some embodiments, the virus is stabilized by a stabilization agent.

For example, the virus is stabilized as described in EP 1496919 B1, by immobilization to a substrate.

In some embodiments, as described in EP 1496919 B1, the virus is immobilized to the substrate via covalent bonds formed between the substrate and the virus.

In some embodiments, the virus is immobilized via its head, leaving the tail free. In some embodiments, the virus may be a bacteriophage.

In some embodiments, the virus is a bacteriophage which targets bacteria in the enteric microbiome. In some embodiments, the virus is a virus which targets archaea which are involved in the production of methane in the gastrointestinal tract of humans. In some embodiments, the virus is a virus which targets metabolic processes leading methanogenesis.

It will be appreciated that viruses capable of inhibiting the formation of methane in vivo in the gastrointestinal tract of a human can be isolated by the skilled person. For example, to isolate a virus which targets archaea, archaea cultures can be prepared in suitable growth media such as 119 Broth. Then, liquid can be harvested from a suitable source of viruses, for example, human gastrointestinal fluid, bovine rumen fluid, rumen faecal material, municipal sewage systems, or environmental samples from sources that provide habitats for Archaea, such as anaerobic soils including peat and peat bogs, thermal springs deep ocean thermal vents. Viruses can then be separated from the liquid, concentrated, and purified, for example by PEG precipitation and ultracentrifugation. The archaea cultures can then be inoculated with the resulting virus suspension and the cultures allowed to grow to allow viruses with specificity for the archaea to initiate a cycle of infection and amplification. These viruses can be filtered from the cultures and passaged repeatedly (i.e., repeating the previous steps with fresh archaea) to obtain pure active viruses. Similar procedures can be followed to isolate other viruses.

Following a procedure as described above, a novel virus with activity against Methanobrevibacter smithii was isolated, which had a sequence as set forth in SEQ ID NO: 1. The isolation method is described in more detail in the Examples and in WO2024/013394 A1, which is incorporated by reference in its entirety. In some embodiments, the nucleic acid of the virus capable of inhibiting the formation of methane in vivo in the gastrointestinal tract of a human comprises at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO:1.

SEQ ID NO: 1:
GTCCTGGTGTTTCTCCAAGGTCTACTTTTATGCTTTCATTTTTCTCTAACCATTTTGTGT
TATCTCCCGAACCGTTTAATTTTTGCCAGCACCCAATCAATACTTCTTCTACTGATTGTT
CGCTAGTTTCAAATGGTTCTAATTTTGGATTACAATCACATACTGTGAGATAATTGAAAT
CTACAAGTGTTGGTAAAGCATGAATATATAATTCTCCTCTTGGTTTTACATAGGGGAATC
TTGAAAATTCAAAAAGTTTTGTTGTATCACATGTTAATGATACAAAATCACATCCTTTTA
GTTTGAAAGTTACGTTGCCGTACCAGTAGTTGAATTTTGATTGTTTTGTGATTACATTTA
CTATATTTTGTTTTGAATCACTTTCATCACCAGTATATTCTGCAGTTATTTCTTTATATT
CTCCTACTTTTTCTTTGACTGTTTTTGTTGCAGTGGTTGTTGTTTCTGCTTCTCCAGGAG
TTAATTTAACTTTTTTACATTTACCATGACCCCATTTGTCTCCTCCACAGTATCCGCAGT
AATCTGCGTCACATCCTCCTTTTTTCATGCTGCAAGTTATTTCGCCTTCGTACACTTTTT
TAGGATTGTCGGTTAATGTTCCAGTTTTGCCACAGTTTGGGCAGTAGTTTTTCCAGGTTT
TTGTGTATCTTTTATATGCTGTTCCTGCACAACAGCCACATGATGGCATCATGTTTACGG
TTATTGTGTTGGGATCTCCTGTTGTGACATTTTCAGTTACCTGTTTTTCTACTTCTTTTT
CAACATATTTCATGAATTTTATTGACTTATTTTTGTAGTCAATAGTTATTTCGCTTTCAT
CCTGAATTAGATTCGGTTGTTGGAAATATATCATTGAAGTATTGGGATCATCATCTGCTA
TTCCCTGATTAGTTATTTCAATTCCTAGTGTGGATGCTCCTTTTATTTTTTTAATTTTGA
GAATAGGGTATGATTCGCTGATATATTTTTCAACATCTAAAATAGTACATCCTGGGATTT
GACGTGGTGTAATGGTTGGGATTTTATTTGAAGAGGTTGTATTTTGTGTGTACATGAATG
GCATGTATTCGTAGTTTTCATAATCTTTTATATAGTGAATATTTTCTTGTACAAATGTTA
AAGAAACAGTGTCTATTATTTTTAGTTCATTAGAATTAAAGAAAACATCTTTTTTTGTTG
TTAAATATTTTGCAAGAACATATATCAAATATTGGAAGCTTAGTCTTCCACCTGTAATTG
ATAAGCTGTTTGGTGCTTTTGTTTGAGTTATGCAGTACGATTTGATTATTCTTGCTAATT
GTATTATTTCATCCTGTTTTATGATGCAGTCAATATGGCTGTGTGATGGTAAATCTGCAC
AATCTACAATTTTAAGTGAAATGTTTTCAGAGTTATTGTCTTCAGTGTTGATTATTGCTT
CTGCAAGTAGAAGTATCATTTGTCCCATGCCTATTTTAACATTGTAGACTTTGTTTTTTG
ATGAATCTTCATATATTTCACTATGTGATATTTCAAACTTGTTTGGGAAGTATTTTTGAA
CATCAAGAAGTACTAATAGTTTTTCAGCACAATCAATAATGTCTTGTATGTAAAAATCAT
ATTCAAATAATGTTGGTGGAATATTATTGTTTACGTCAAGATTGTAAAATTGTCTAAGTA
TTTTTGAATAATCTACTTCATCAGTATTTTCTGAAGAAATTACTGGCATTATACCTAATG
CATTTTTGGTTTCATCACTACTATATGATAATTCATTAGTATTTTCTCCAATTACAACAC
TTTCTACAATTTTATTGTGTGTTACTCCATAATTTGCAGGTTTTAGTAATTGTATTTCTT
TTATAATGTTATTATTTTTTAAAGAATAATTTGTTTTAAATACTAATCCTGATTGTTTTT
CAATAGCTTTTAATAAGTTGTATTTTGTTATTGAGCCTTGAACATTGATTAATCTTTTAC
TATAATCAATACTGTCAATATCTGTTTTTGTAACATTGAAAAATCCTTTCAGTAATTTGT
TTAGGAATAATTTACTGATAATGATTGTATTTCCATTTACATGTGAAGCATAATTGGGAT
CTCGGATATAGAATGGTTTACAATTGTTTAATTCTACAATTATTTCTTCTGCATCTACAT
CAATAGATTTGTCTATATAATCAAAGGTGCATTCACTATTAATAACAAACAAACAATTAT
CAACAAAGATTTTGTTTCCTTGTTTGAATAATTGCTTGTCTCTTTTAACATTTGTAAGTT
TATGTGATAGTTTTAATGTTTCATATCCTTGATATTCATCAGTAATATGTATTTCTGATA
ATTCAGTATCTAAAAATTGAAGAATATTTTCTTGATTATCTAAAACAACAACCATACTTT
TTATCCTCCTTTTTCATAGTAAATTACATCAGTCACTTTACAATTGATACTGTTTTCAAA
ATTAAAATGATGATCCAATATGAAAAAACTAGAATCTACACTAATACAATTAGGATCTAT
CTTGAACCATTCACTATATTTTTCATAGAATACATTTCTATTTTCACAATCAATTTTAAT
CTTTGTTGTTTCAGGTAAGTTTGTTATAAACTCTCCTTCAAGTTTCATTAGTTGTCCAGA
TTCTGATTCAATTATTTGTATGCTTGTTGATTCATCTTCACTTAATTTATACAAAATAAT
GTCTGGTTTTACTTTTCCAATTGTGGTTATTGTTCCTGCGAAACTTTTTCTTAATGGGTT
AATATTTCTACTTAAACCACTAGGGATTATTAAGTTCACTTCACAATCATAACCTCCAAC
CATTGCTTCTGCATCAATTGAATCTTCTATATAGTAATCAAAACATTCTTCAGGTGCGAA
AAAGAAACTAATACTTTTTAGAATTGGTGAATCTAATGTGTCTCTTTCAGGATACAGGTA
GTCACTTACATATTTCATGATTTCTGTTGATTCTTCAAAATCATCTCCGTAAACTCTGAA
TTTTAATTTGATTTTTTTATCATCAACATTGACTCTTATAGGGTATTTCCCATCTGCTCC
TTCAATTTTATATTTTTCTGTATCAAAATTAGCTCCACGAGGTATTTCTGTTTCAGGACT
CATACTTATAAGGAAATATTTGCAACTAACACCATTAATATAGAAATCGCAGTCTTCTCC
ACTATTTTTTGAATAATATACTGTGATTTGCACATTTTTCATTTCGACTTGGACTTGAGT
GACATTGTCAAATGCATCATCAATTTGTAAGAAAAACCTTAAATCATTTAATATTGTGTT
GATTTCTGGAAATGGAATGCCGAAATTATGGAATTTTCCACCGAATTTAAAGTTAGTGTC
ATTTCTAGTGATGTTTATGCTGTCTAAATAGTAATCTAATTCATCATCATCTAATGTTCC
AACACCACAAATTAGATTAATTGATTCTTCAACACTTATGTCTCCAGTTATTTCTATTCC
ATGAATCAGTACATTATTGTTTTCTAATCCTTGCCAGTTGAAACCTTCAAAGAAATGTTT
TGTAGATAAAGCTAACTCTGGAGGTTCAAGTAATAAATTTGCAGTGCTGCCTACATCACC
TAACAAATATCTTTTTGGAGATAAAGCCAATACTGGATATTCATAACCTGCATATCTTTC
TTTAAGTATTAAAGATAAATTACCAAATTCTACAGTTCCATAGTCAAAATCTATGAAATT
CCCATAAAAAATTAGATAGATTGGTTTACTTGCATCATATGTAAACTCACAGGATAACTC
TTCCCATATGTCCTTAACACTGATTTGATTGGTGTATGAATTTGTTGTTCCATTTACAAC
ACAAACTCTTAAGTTTTTCAAACCTTTTTCAATTTTAGAAGCATTTAACAGTCTTCCTAA
TGCTGAGAAAACATAAGTTTCACCATGTTCAAGGTTTGATAATGTGAATTCTGATAAATT
CAATATTGAATAATAGGATTTATATGAATCAGGAATGTTTACTTTCACTTTATCTGTGAA
AACACCTAATAATAATGGACTGGTCATTTCAATATTATGCTCTCCACTACTTTTAGAGTA
TAAAGGAATATTTGTATCAAAAACAATGTTTCCACTATCATCTTTTTGCCATTGTGATTT
TGAAATTTGTAAAATATTATTTGATAATCCTGGAACAGGTTCTTTTACAGTTAATCCTCC
AAAATTCAGATTTAGTGTTAAATCTAATGGAGCATCAGTTTTTAAAGTTATGTTTAAGTA
TGTTAAATCTGTAACTGTATCTGTACTAATTTCAATTTCTTTGTTATTTGCATAGAAATC
CCATACTTCCAATTGGGGTTTTTCTACTACAAATGTTGTTTTTGTTGAATGGCCACTATT
GTTTTCATCAAGTGTGATTGTAAACAATCCTTCAGATGTTGGAGTTACATAGAATATTAA
TTCTGCTTTACCATTTGTTGTTGCTTGTCTCCATATGCCTGTGGTCCAGTCATAATATGG
TGAATCATATGTGATTTCACAGTCTCCATTTGTGATTATCTTTATGTTTGCCGATATTTC
TTCTGAAGGTTTTGTTCCACTGGTTTCAACCATTGTGCATTTTACAGCTACTTTTTCACC
GTAACTAACAATATTTGGTTCAATGCTAATGCTTGGAATATAATTTCCAGTTGTATACAC
TACAATTACTCTTAAGTATTTCATTTCAATGTAACATGGGTTTGTAGATGTATTTTGTGG
TAAATCAAATTTAACATCAAAATTTGGAGTGTTAATGTCTTTTCCTTTTAGATTCAAATT
ATTAAATTGAACTTCATATTCTCCAAAATTATTTCTAGGTGGGGCATTACCTGTTTTTGA
CAGGTTTAAACTACTTAATATGATTTCTGGATTTGAAAAACTTCCATGTGCAGTTTGACT
TATCTGTGCAGAGTATGCTAATTTTCTATATGAATATTGTACTATTATACTCGTTACTTT
AGCTGAATCTGGAATTAATCCATCATATTTAAAATCATATCCACGTATGGTTGATGGATG
TTTTCTTGAACCATTTCTTCCAGCAATCGCATTTTTATATTTTGCTCCAGGATCTGCATA
ACCCCAATGTCCCACAGTATTTAAATCATTTGTAATATTTTCAATATGATCACATGAACG
ATAAGGTATGTTGTTTCCTTCTATATTTTGAACTTTACTAGGATATCTTAAAATACTAGG
CAATTTAATAATCCCTCCATCATTTTTTTTAATTTTTAATAAAACCATCATACTCTGAAT
TAAACAATAAACAGTTTAATTGAAAGCTAATGTAATCTCCTTTACTTACCCCATCAGGAA
AAGCAAATTCAACACTCACATATTTAGCACCAGCAGGTATTTTTGATGTTTTAATAGCAC
ATCTTCCTTTATTACCCATACCTTCATTAGCAATATCCATAATTTCCTGATTTTCAATTA
ATTTACCATCTTTATCGAAAAATACAAACCCAACAAGTATGCTTTCAATAGTGTTTTTTG
CATCATAATTAGATGAAAACACAATACTGGAAGCATCATCAGGTATGTGTACTCCTTGAT
TGTTCTGATGGATTAAAAGAAAAAAAGGAGAAGTTAAATCAACATTCTCCGAGAGATATG
TTTCTTTAACTTTCAAAGTATAACCATTTCTAAAAGGAACAACTTCAAAAAAATCACTAT
ATGGGTTGAAATTAAAATCAAAATGTTCACAAACAAACAAACTAGATAAACTAGATTCCA
TACAATATTGAATATCTTCACTAAATCTGTTAGTTAATGAAACATCTTTACTTAATTTAA
ATGAATATACATTGTAAGATTCCCATTCATCAGGTTTTACATTATTTAATGAAACCATAC
ATTTATGTGATTCAAAATCGTTTTTATCTTCCAGATATCCATGTTTGTTACTTATGATAT
TACCGAAGATTTCACATTCTTCAGGATTAATTTTTATAGTTTCCATTATCTTTTAACCTC
TCATTCTTTTAATACGATTGTCTCTTTTAATTAAAGCTTCAGCAATGCGGTCAATTAATT
TGGAATCAGTTATCACTTCTTTTAAAGTTGTGATTATTGATTTTTCATCAATATCTTCTC
CTTCAACAGTAACATTAAGATTATGATCTATTGTTAAATGCACATCATCAGATGTATAAG
ACACTAAATTATCAGCAGTAGTATTATTCCTATTATGTTCAATATTCTGGAAGTATGCAT
TTTGATTTGTTTTCATTAAATCAATAATATTATATATTCCATCTGTAATGATTCCGAATA
CATCTTTAGAACTTCCTCCAGGACTTGCTGAACCAAAACTAACTCCAGAACCTCCGCGGA
ATATTCCTCCACGTTTTTGGAATTGTGTCATATCATATATTTGACCATTAACCATAGCTG
CTACATGGCCTATTCCATTCCATGATCCATGAATCATGTGTCCTGATAATCCCAACATAG
ATGCTATTTCAATTATCATTTCTGCACCATCAAAACAGTTGCATCTTACTTGATCCCAAA
CTTGCTGATTACTATATCTTGAATTATAATAAAATTCATAAGTTCCAGGGTTTTGGAATC
CTCTTGCAGTTAAGATTTTTCTTAGAAGATTTTCAAAGTTTCCAGGATTTAATTGCGGTT
GTTTTCCATCACGGAAATCTTCCACATGATAATCTGAAGGTATTTGAATTCCAAGGAACC
ATGGGTCTGCTATCCTCCATTTGTATGCAGTATTCATTATTTTATTCACATTAGTATCAG
GAATTCCTGCAAAACAAGTATTTGGATTAACACAACCATTAACTCTTGCCAAATCATTAA
TTACGCTTGTTGGTAAATTATAATCTAAAAGATTTAACCATGATTTGCTTTTATTGTTTT
GAGGATGTTTTACTTTAGAATTAGTGTCAATTACTCTGCCTTTTCCTTGTCCTAATCCTC
CAGCAGCATAACTTGCTATTGTGCCTTTTGTGGATTTTCCAAAGTTAATGCTTCCACCAG
GTAATGTTCTGCCTCCAATTAATCCTCTTGTTGAATTAGTTGCTGGGCCTGCAGGCAGTC
CACTACTGAAATGTGCATTGGCTAATTGATTATAGAAACTTGCAATACTTCTATGAAGGC
TGCTGAATTTATTGTATGATTGTGTTTGTATGCTTCCTGCAGCTGAAACAATATTGTCTT
TCATTACTCCCCATGCATTAGTCATTTTATTTGTTACATCTATTGTGGAGTTACGTACTT
GATTTAAGTTCTGGGTTGTTGTTGATTTAATATTGTTCCATGCTTGAGTATTTGAATTAC
TCATGCTTGTTAATGCAGTGGTTACTCCATTACGTGTTGTTTGGAAGCTTGTTACTACTC
CTGCAACTCTCATTTGAATAGCTCCTGCATTTAATGCTATTCCTGCAGTTAATGTTGAAA
AGCTTGTTCCGATCATATTGTTGCTTGCAGTGATGCTTTCACTAGCTAATGATACTTCTT
CAACAAGACCATTATATTGGTCTCCAGTTTCTTCTGTACCTGAATTATTATTGCTTAAAT
CAACATTTGCACTATCTGTTTCAACTTCAGTGTTTGTTGTTACTGGAGTTGTGATAGTTG
TTGAAATATTATTTAATGCATTTTCAACATCAGCAGTATTGAATCCATTTACAATGTTTT
TTCCAACATTTTTACCTGCTTCATATGCTGATTTTCCTTTGTCTTTAACTCTTTGTAGCA
TGTTAAAGAATTCTAGTACTACTTTTTCTTGGATTATTCCTGGTGAATGTATTCCCATTG
CGGAAAGCATTCCGTCTACAATTTTTTTACCGATTTGCTTTGCATTTTCAACTAATTGTG
AACCTGCAGATAGTATTCTTGAGCCTATGTTTAGGAATTCCTGGTATACTTTTTGAGGTA
ATTGCTTTATCCAGGACATTACTCCAGATACTACTTGTGATGCTTTTGTTTTAGCATTAC
TTACCCATTGTGCTCCTGCAGTTATTACTCTTACAGCAACTGCTAAAAGATACATTTTTA
TTTTATCTGGCAATTGTCGGATAAATTGAACTACTCCTGTAAGGAAGTTTCTACCAGTAT
TAACTGCATTATTCCACATTGACTGAGCCCAACTAACAACACGATTAATCACATTTGATA
ATATTGTGCTGATTGTTGATTGAATAGCTGCCCAAGCCCAAGCAATTGCTTGTTGAAGAC
TTATTTGACCAGTAATTAACTGGCTAAAAATTGTTGCCAGTTGCATGAATAAATTGAATA
ATGGAGTTACAATATCAAGTACAAGTTGCCATGCTGGACCAAATACACTTAATAAGAATG
ATGCAATTCCATTGAAAACATCCCATATTATGGTGAGTATGTCTATTGCAAGTTGAACTC
TAACTTTGAAACTTTCAAATACTGCTCCTATTGCATCGATAACTGCTCTTGTTCCATCAA
CTTTACCTTTTGCACTTTCTGGGAATATTTCATCCCATATTCCTTTCAACCAATCTACAA
CTGGTTTGAGAGATTCATTCAAATCAGACCATGCATCTTGAATTGCTTTGATAGTTGCTT
TTACATCAGGATGATTTATGAATGCATCCCATAATCTACCAATATTATTCTTTATGGCTT
CGAGCATGGTTCCTACATCTTTCCACCAACCAAAAGCTTTACCTACTTCATATATTGCTA
CTGCTATTGCAACTAATGCCACAACAACCCATGTTAATGGATTTGATAAAATTGCTCCTG
CAACTGCTAAAAAACTAGCCGCCAAACCCATATTTGCTATTTCAACACCTAATGCTCCAG
CAATATAAGTTACAAAACTAGTCGCACTAGCAGCTATTGCGGGAATTACTGAACCAAGAA
TTACAATACCCAACCCAACTAAAGCTGTTGAAAGTACACCAATAGTTACTGCTGCTTTAG
CCCAATCGGGCATGCTTGACCATGCTGATTGTAATGCTTCAACCATCCAAGTAATCATTT
CAACAACTGTTATAATTATGGGAGTTATTGGGACAAGGATAGATTGTATTAATTTGCTTC
CAGTTACTGTTAATGCTGCCCAAGCATCATCTAATGTTACAATTTCTTGTGCGGACTGTT
CAAAACCCATATCGCTAAGTGTTTTGTTCATTGCTTGGAGTAATCCAGTTTTATTATTTA
TATCTCCATCCCATCCATTTTTCATGAGCATGTCTTGAGATATGCCTATTTCCTGCAGTC
TTCTGAATTGGCCATCCATTGCATCTGATACTGCAAGTATTGCATCTTCTTGTGTTCTTC
CTTCTTTTACGAAAGCTGAACTCATTACTGCAGTTGTTTTAGTAAGTTCATCCATTGAAG
CTTTAGGAAGTTTTAATTTCACTCCCATTTCTAATGCGGCTGCACCTACTGCATTCATAT
CTACTTTTCTGAAGCTATCTTGCATTTTGTCAACAGCCCCATGGAATTGTTGTAGTTCTC
CTTCAGTCATTCCTAATCTTTGACCGAATCTTTCAAAACTTGCAGCTGCATTTATTGATT
CTCTTGCTCCTTGAACCATACTGTTTACAAGGTCAAAACCAATCATTCCTACAGTCATAC
TTGCTGCTGTTCTAAGAAATCCAAGACCTCCACCTGCAGTTTTAGCTCCGTTTTCAAGTC
CTCCGAAACCAGTTCCTGTTTTTTGTGAACTTGTTCTTAAACCTTCTAAAGCTAATTGTG
CTTCTCTTGCATCTCTTGTAACATTATCTAAACCAGTAGTATGGAAGTTTAGCAATGAAT
TTACTTGTTGAAGTATACCAATTAAAACTAATAATATTGCTTTTAGTAGTTCTGCTGATG
CTGTTGTTCTACTGAATCCCGAACCATCAATTGAATCTAAGGCTGCATCTGTTGAATGTG
CACTTGTTCTAGCTCGATCTAATGCCATGTCTAATTGTGCTGCACTTGATGAAGTTGCTG
TTAATGTGTATCCAGTTATACTATTTATTCCACTGCTTAAACTAAGTGCACTGGATGAAG
CTTGTTTTAAACTTGAAGATAATAATCTGGTACTATTATTTGAACTATTCATCGAGGAAG
ATAATCTTGTAGCACTAGATGATGCTGTGAGTAAATTACCTGAATTAATTGTGTTGATTG
AAGTGTTAAGTCTATATGCACTATTCATCGATGTATCCATTGAAGTGGTTAATAGTTTTG
CAGAATTAGATAATTGATTAATTGTATTGGGATTGATTGAATTTAATGCTGTTTTTGTAG
AATTAGTGCGATTTGTAACTTCAGTTAATTCTTTATCTAACTGATTAGCAGAGTTTTTGG
CTTTATCCATACTTGAGGAATCAAATTGTCGCATTGCATTTTGAGCTTGTTGTGCTGCTT
TTTGAACTTGCTCCATTTGTGCTTGTATCTCTTTTAATTGAGCTTTAACACGGTTTTCCA
GTTCAATTATTAACTTTACAATATTATTGCTCATTATTCTATTCCTCTGCTAATTTCTTG
TTTTTTTCTTCTGACACGGTCTTTTAGAGAATGTGAAGCATGATTTGTACCTGAAGATTG
TTTATCCTGTTTATCACGAACTAAGGGTACTGCATGATTCAAGAATAATTTCTGCCTAAT
AGTTAAATCTGCTTGGTTTTCTGCTAACATATAACCGATGTCTTGAAGAGTTACTACTTC
AATAGCTATGTTTTCATTCTTCTTCACGAAATTCTGCTATATTATTATCCATTGATTTAA
GGTTATCTAAACCACTGAAAATTAATGCTTCATTTACAATTAAGTCTAATGTTCCTGCTT
TTAATTGTTCAAGGTCTTTTTTGGTGAATTTATCAGGATTGTCTGCATTGTCTAATACTT
TTAATGCAAGGTTCATTTTTGCATCATATTTCTTTTTTTCCATTGCTGCGAGACTGGTTT
TGAATTTTGTGGTGTCTTCTGCAAGGCTGGTTTGTTTTGCTTTGATGGTGGAAATCATGT
CGAAGTTTCCTACATCACGGTATGCTGATGTGAATTGGTTGTATTCAAGGTCGCTTATTT
CTCTTATTGCTACTTCATTATCGTACATTGGTAGTTCGATTATTTTTGTGTTTTTGATTC
CGCCGATGATCATTTCTTTTGTAAGCATAAATATCACATTTTTATATAATAATTTTTTTT
TGAATTTTAAATAAAGTAATTGAATGAGAATAATCATATTCTCATTTCATTTAATCTGTG
TAAACAGTGATTGTTTTTGTTTTACTTGCACCATCAATTAAAATATGTGCTGGTTCTACT
TTAGTTGCAGTTGGTTCACCAGTCAACTCTATATCATACACTCCATCTTCAGCAGTAAGT
GTTACTTCACCATTACTTGAAGATTCAGCATCAACAGTTGTGGATCCTTTCATTAATGAT
ACTGTGTAATCAGTTGTTAATTTAGCACCTTCCTTATCAACAAGTTTTATTGTTGCATCA
TAAGTTGGGTTGGGATCACTGTCTGCTTTTCTTGCAAATAATGCTCCAGCTAAATTTTTA
TAATGGTCCATTGTTGATTCAACTGTTTTTGATAAACAAACAATACTTAATTCATATTTA
ACTGGGTCTGCATTCATTGTTGCAGATGGTTGGTTGATAATACACATTGGACAATATAAT
TCGAATTTTTTATCACCATTTCTAATGTAGAACATTAATTCAAAGTATTCTCCTGCAAGG
TATCTTCTTGGTCCTTCCTCACTTCCCCAGTACATTTCCAAGTATTTTTCATCAGTATCA
TCTACTGTTAAGTTTAAACCAATTTCTCTTTTAGCTGCTCTTGGTATTTTACACATAAAC
CTTGAGCCCATTCCTCTTGCATCATCAGTATTAACATTGTTTTTGATTTCAAGACTTATT
TTATTGGTTTTACATCTCATTTCAGTCCATTCATCTTCACCAAATCTTCTCATTTTAACA
CTGTCTATATGGTAAAATGATAATGGTAATTTTCCAAAGTTAAACTCTGATAATTTTTTT
AATGGTTGTTTACTATTGATTTTTGATTTGATTTCTGCTGATGCAGTTAAGAATTCTGAT
TCTACTTCAATACTTAAACTGTCCATCACCATACCTAAAATTTCCATCTCGTAGGTTGCA
CATCCTGCCATCACAGTATAAGAAGGTAAGATACTACTATTTGTACCGTAGATAATATCT
CCTTCTCTTCTTCCCAATACTGCTTCAAGAAGGTATTCTAATGTTTCCATTTGTAAGTTG
CAACTGAAACTGTTTTCAGGTACATAGTAAGCAGCTACACCAGATTGATAATCCCTACTT
ACTACTGAATCATATTTTGTAAATTGCTCTGGTGGAGATACATCCATCTCTGATATTTCA
ATATCAATAGATTCTCCACTAAGGTCTTTTTTTGCAAATTCTCCTTCTTTTACTAATCTT
AAATATTTTAGTTCTTCTGCGGCTGTTACCATATTCTTTAGCCTCCATCACATTTTGAAT
TCTTAATTTTAAATCTTATTTGGAATGTTACACCTGCACTATACACATTTTTCTTTTTAC
CCAGACCATATGGAACCCATCCGAAATCCTTGGTTTTAATATTGAAAAATTCTAAGTCTC
CAAGTAATCTTGAACTTATTATTCTACGTTCTGCCTGTGAAACTATACTTGTAGCTTCAT
GAATACCTATCTTAGGATTTTTATTGTTAAGCACCTGTGAGGATAATATTATTTCTCCTT
CAAAGGTTTCACTTAAACCAGTATCAGTAATATTTTTAAATGGTTCATCTACCCATAATT
CCAGATAAGGTAATTCTGGTGTTCCCAGACTTACACCCATAAGAGATAAATGTTTAATAG
TGCCATCTTCAATCATTTCAGATAAGACCAGTTGGAAACCTTCCATTAGTTTATCATAAG
CTTCTATGAAATCTTTCATAATCCCACCTCTTTTAATGTTTCAGACACATATTTGTTAAT
TTTTGATTCAGTAGTTTTAACTGCATGAGGATAATAATGGTATCCCCTGAATGCTGCTAC
TTTTTTAGGTTTAGGGACAAAAACATCTTTACCAGATTTATCCACCCAATGCAGGGCTTT
TTTATTTTTAGCTGAGAAACTTCCACGACCATCATGAACATATTTTTCATAACCTGAAGG
TTTTCCTCTTGCTTTTATAACATACATTTCATCTGTTTCCCTTCTTGCAGTTATTGCAGT
TAAAAGATTGTAACGATTGTATTTTATCCTACTTTGAAGGTAAGTTTTAGCATCTTTACT
TGCTCCATAACTTACCTTTTTACCTAATCTTGGAGGTACCCGTAATATTTTTTCTTGCAG
TCTTTCCCATCCCGCATAATCAAAATCAAAATGTATCATAAAAAATCATCAACTCTTATA
AAAAATTAAAAAAAATGTTTAGATTGAGAAAACATGAATCTTAGATTTTTTAATGTAAGG
CTTCATTCTTTTTTCAATGTCTTCTGTGAAAATATTGTTTACTGCCTCATCAAAATCAAA
ATTTTCATGATCAGTAATTCCCAAGTCCTGTCTTACAGCATGGCTTCTAATAATGTTACT
GGTTAATTCAATAATGATTTGTATTACATCATCAGGAATTTTATCTGGTATATCATATTT
CTTTTTAACCCATGATTCCATTGCAGTGTAATAATCATTAATATAATTATCTAATTCAGT
AGTTTCTAAATTAAATAATTCTTCAGTATTTTTATTAGCTCCACTGTACTGTTTGATTTT
TTTTAAGGTGTTTTCATCCAACATAAATTATTCCTATGTTTTAGTCATTGTAATGGTTAA
TGTAGTTGAATCATCACCTACAGTATAATTACCTGTGCTTTCAAAGTTTTCATAACCAGT
ACATGTTGCACCATATGAATAAACTCCATAAGGAACATTACTTATTGTACAGCCTCCAGC
ACTACCAGTACCGTTACAAGTGTAAGTTTTACCATCAGTAATATTTGTTAAAGTTACAAT
AGCTCCTTCAACAGGATTTGTTCCATCATCAACACTTACACCAATATTTTTAGTACTAGT
TGTTGTGCTGGAGTTTATGCTTCCTTTATGTATGCAAGAGCAGATGCATCAGACCATGCA
AACTGAATATCAGCATACATTGTAGAAGCAATATAATACTTGTTAGATTTTAAATCAAAT
TCAGATTCAACAATAATATTATCAGGATCCGCCAACCATTGAATGTTTTCTTTATGAGTT
AAAATTACTGGTTTTTTAGTGAAACCATTACGAAGAGTACTGAAAGCTGGAATTGGTACT
AATGGTACTTCCTCAATAATGATGTCCCCATCTTTAGTAATAACTACATCATTAACAGCA
TACTTGTCATGATTATCTGCTACATATCTCCATACTGCTCTTTTAAAACTGTATGGTACA
AATGCAGCTACCCCACCATCATTTTTATATTTATCAGGGAATAAATCCAACATTCTACGG
AATTCTTTTAACGGATTGGAATCTGTTGCAGTTAAATCTATTGTTTCTTGATCTATATCA
GAATCATCTTCTAATTTTTTCAAGATACCATCATTTACTTTATATGAAGTTGCAACATTA
GATTCAGTACTTGCAGTATTCCCATAGATTAATGTTCTTTCTAATGCTCTACCATTTGCA
CTACCAAATTGACCAGTTAAAGTATTCATGAAATTCTTTTGCTCAATACTGTCATATAAT
ACAGTTCTGTGAACACCAGTTAAAGCACGATATTCTTCTGCATCAAAAGCCCTATTAGTG
AATGTTGGGTCCTGTTCATTGCTTAAAGTTTGTGGTGTACCACTGATTCTTCCAGCTTCC
AATTCAATATCAAAACTCATCATGTCCAATTCTCTTTTATGATTGTGAGTTGGAACAACT
TTAGTCTTGTTTAAAAATGCTGTTTCTTCTTGAACAGCTTGCATGTATTTATCTGCTTTT
TCCGCTTGAAGAACTCCTTTACCGAGTTTCCCACTTCCTTCTCCAATATCTACAAATTTA
AGGATAAAATCTTCATTATGCATGATTTTATCAGCTAACGCTTGTTCTACTTGTTTAATT
GTCATTTTATATCAAACCTTCTCATAAAAAATTTAATTATTAAGTATAATATCTTCTTTT
TTTCTACCAGGTCATTCCATTTGAATTCCTACCCATTCTCTCAAGGAATGATTTTTCAGA
AGCATTGCCTTTTGCAAGGTCAGGATCAATACTTTTACTAACAGTAGCATCTGGATTAAC
AGCACCATCAGCAGTAATAGTAGTATCCACTTTAACTTCTCCTCCATCATCATCTTTTGG
AGGTTCTGATTTTTTAACAGCTCCAGGAACAGGATCCTCTTTTGGAGGTTCTGGTGGTTT
TTCCCCACCTTCAAGTTTAGTTATCCTTGCATCCATAGTATCTAATCTTTTATCGATTTT
ACCTAGTGCTTCTAAAATTTTATTTTCATCTTCCACAGGTTCATTATTTTGTGCTGGAGG
TTCAGTTGGTTCTTGAGGTTCAGATTTAGCAACTAAACCATTAATTAATTTTTCAAGAAT
ACCTTCTGAAACCTGCACATTATTTTTTCCATTGTCTTCTTTTGTCATAACTTCCACCTC
TTCTGGTGTGATATATTTTTTCACAAATTCTTCATCATCTTCATATACTTCAAAAACCGC
CATAGGATGTGATGGTTGATCAACAATACTGATTGTTGATGGTTTCCAATCTTTAATGTC
TTTAAATTTCAAAGCCATATTTAACCTCCTAATCCTCTACTCATTTGAGCAACACTTTTA
TATGGTGCAGCCAGTATGCTGAAACCATTGTATTCCCCATCTCGGATTGCTTGTTGTATT
TCCTCATCAGTTACATCAACAGAAATAAACCAGGATCCTTTGGGGTAAGTGTTTCCCCTG
AAAGTGGTTGGTGATTCAAGAATATATGATTCTAAAATTCTTCCTACAGGTTGGAGTGTG
TGCTGTACATCAACTCCTAATCCCAGGCGATTGTATATTAATGATGCTTGTCTTATTGTT
TCTTCATCCAGTATGTCTCCTGTTGCATCAGGGATTCCTGGTATGCAGACTGCACCTTTC
ACAATCATAGTTTTTTTTGACTCCCCATTTTTTTATATTGGAAAAAAATTAGTTTTGTTG
GAGTTTTTTTTAGTAAAAAAGAAAGGAGGATTTTAAAAAAAGATAAATGATGTTAAGTTA
CTAAAATAAAAATATTTTTTTATCCCCCTTCAATATATAGGAGTGGAAAAATAGAAAAAA
ACGAAAAGTTAAATAAATTTATTCAACAATTTTGACCATAGGTTTAAATTGAGGATATTT
ATCAAGAAAATCTAAAGCATATTGTATATGATATTCCCTAGCTTCTTTGTGTTTCCCTTC
ATATTTTAATCTATGAGTTACAGCCCGTTCATTCTGATCCAAAGATTCAAACTCCGGATG
TTCAATACACTGAATTAATCTTAATCGATTAATATTAGGAATAACTTCACGTACAATACT
CATTAATCTATGAGGTAAACCTGAAGGTGTTGTAATGAAATCTCTTAATTGATCCTCAGA
CCAATTTTTAACAAATTCATAATTAGGAATCAATTTTTCAACCATGATAATGAACCCCAG
TATTTGTTAGTATATTATATATAAAACTTCCTATATAAAAATTACGATATCATTTCCCAA
TGAGCAATAAACTCTGATTTTTCACGTATTAATCTTAAATCAACATTTTTATCAAACAAA
ATTTCAAACTCCTGAGGAGTTTCAGTTATTCCTTCAATATACAGAATTTTAATTCCTTTT
GGAATGACAATATAATTAATATACTCCCCATATTCTTCAGGTTTAACATTTTCGGAAATA
GAAGTTGATAAAAATGAATCTGAATGATAATTATCTCCTTCTACAAATTTAGTCATTGGA
CTTTTTTGTCTTCTAACTACAACAAGACTTTCTTCTAATATTTTACCTTTCTTAAATACT
GATTTTGAAGATTTAATTGCTTTATTAAAAGAGATATAATATTTTGAATTAGCTAAATCC
TGCTCCCATAATTTAGAATATTTTCTTTTAATACGCCACCTCTTAACTGGGTTGAGCTCC
CCTTTTATTTCTCTGAAATAATTATTCAAACTATAATATCCTTCATCAGAATATGCTTTT
ATGAATTTATACTCTTCAGAAGTCAAATCTTCTTTTTTAATTCCATATTTATATTCAAAT
GCCAATTTATCTTTAGATGATTCACCAACACCATAAACTGTTTTATTACCAATTTTCACT
GCTTTATTTTTAGCTTTACTAAATTTAGTGATAGATTCATTTAATTTAAATTTACTGAAA
TCTCTTTTTGAATTTTGTTTTGGTTTTGAAGAAATATTAATCATTACTGTTTTTCTTTTA
ACATTTTCAGTGATTTTACTTCCTGTATTTTTAATCTTGTTACCTATATTCGATATGGTT
CTAATTATCTTATTCTTAATCTTAGAAGTTATCTTCAAAGAATTTTGATTACTGCTACTT
TTATTCTTACCATGTAAGTAAGAATTAGAAGGAATATTAAAAGAACTTTTACTTTTAAGA
TTACTTGGAGTACTATTAGTATAGCTTAACCAACATCTGCAATTAGCAACATTCTCAGCA
CCTCCAGCTAAATCCCCAGGATACATCATCTCAGTACGATAACTACCAGTAATAATAAAA
TAATCATCAATAGGAACACTCTCAATAAAGTTAGCACGATGCCAAATTCTATGTTGTCTA
GCACGACCATTCATCCAAACCTTATACTTATACCCCTTATCCAAAGCATGTAAAAAACTC
ATCCTAGACTGATTAGTATGCAAAGCATCACGAACAATATTCTTAGCATTCTTACGATAC
AACTCCCCATACTTCTTTTTAATCTCACTATCAGACAACTTACTTTCAGCTTCAATCTTA
GCTTTTAACTTATAAGCATCACTATCAAAACGTGAAGATAAAATCTTAGAATAATCCCCA
ACAGTTTGATCAACACTACCTTGAATAATCTTAGAACGCTTTTTCATTAAAGTATTAGCT
TGACCTTGCAAAGCAGTATCAATTATGTTCTCTGCAATCTTTTTACTATTCCCAATTTTA
TTTCCATCAATAATATTATTTAATAATTTATCTTTATAAGAATCAATACTTTTATCAGCT
TTATTCGTATGTTTAAACAAATGATTAAAACTTATATCTCTTTTAACCACATAAAGCTGA
TGCAATCTTTGAGTTAAAAGCAGATACTGAAGTTTCTTTTTAGAAATCATAATAAACCAT
TCTTATTAAAATCAGAATTCAAAGCATCAATCATACTATCCAATTTACCTTCAGCATCCA
TTGGAGCATCACCATACAAAACCTTATCCAAAGACTGATTATTCATAAACCTACAATTAT
AATACTCATCATCTTCACTCATTTTCAAATCAAAAGATTCACCAAAACGATTAACAAACT
CCCCTAAAGTCACTGCACCATTTTGCAGTAATTTAATTCCACGATCCAATACTTTATCTT
CCTCATCAAAATTAACAGGCAAATACTCAAGTTTATGTGTAAATATTCCAAATTCCTTTT
GAATAATAGTCTTATTAATCAAATTAGCCATCCTCTTTTGCAATGTAGCTACTGTAGATT
TACTATAATTTTTAAGCAAAGTTTCAGTACGATTACTAGCAATACCTGTAGACTCAGAAT
CACCTAAACGTTCACGAGGTACACGATGAATACGACGAATACGATCAGCCACACTACCTG
ACAGTTCTAAAAAACTGCCTTCTTTTTTCTCATCAGCAATTTTAGTAACATTAACACTAA
CATTATTTTCTTCACTGGGAATAGTGAAAACTAAAGCAGTTCCAGGCTCATTGGACACTT
CACGAAACTCATGTTCCAAATCCTCTTCAAAATCATCAAAAGTATAATCTTCTTTTTCCT
CAACATTACCTGTAACAGTAATAATATAATTCGGAATACCATGTGCTTTAAAATGCCCTT
GCTGATACTCAATAATAGCATTATCAGTTAAAATAGCATCCAACTCGGACAAATATTTCG
GTCTACCATAAACAAGACTTTCATTACTTTTAAGATTAAACCAGATTAAATCATTTGCAC
GATTTTCATCTGTAATATTATCATCCCACAAACCAGTTTCACGATTCAATTGCTTAATAT
TGTCAGGATCATATAATTTGAAATAATTTTCTTTATGCCCTATCTTTTGTACAACACGTT
TTTTATCACGACACATTCTCAGGTATAAACTGCTTACATGATTAACACTTTTCAACTCCC
CTTTTTCCCTAAGTAGTTCTAATCCTGCAAAACCAAAAGCTTCTAAATCTTCTAGGAATG
ATTCTATTTCCTCATCAAAATTAAAATCATTTAAAAAATCATCTAATCCTTCAGGTATTT
CTTCTCCTTCTTTAGAGATTATTTTTTTACCTGTGAAAATTGCATCTTCACATTTAACAG
TAATACAAATATCATGCAAACCACTAATATCACGCAACTTATCTAATTGGAACGGGTCAT
ACGCTGGGTCAATTACTTCTGTACCATATGTTAATTCATCTTTACTCATTTCTTGAGATT
TAATCTCATATTCATTTAAAACACTTTTAATCACTGAATCTCTTAAAAAGTTACTTTTAA
CTATTTTAACCATTATTAATTTCTCCTTCTACGATTTCTTTTTCTTAAAACTGTAGGTTT
TGTTTGAGGATATAAACCTTCATGTAACAATGATACACTATCTACACGGTCATCATGTGT
TGTTTCATCATCAGCTATTATTTTCTCTGATGGGAATTTCACAGCTTGTTTCATGAAATC
TTTCAACCATTTACCACGAACAAATAATATCCGTCCATTATTCATCCCCCGTATAGTTCT
ACTTGCTCTTATTAATTTTGATTTAGGAACACGAATAAAATTAGGATGATAGTCTTCGAA
TTCGTTTTCCCAGTATCTTTTAACTATTTTACCTGCAGCTGCAGGCTGATACTCAATCCA
ATTATCATACTCTGAATGTTCATCCATAATCCTTGTCATGTATTGTTCTAACTTACCTGG
CTTTTTTTGTGTGCTTTTCTGATTATGCACTACACCTACTTTTCCTTGTAATACTGTTGA
AAAACTACATGCTGTATAATCAGAAGTACTTTTTTCTGTTGCTGCAATATCCCATGTAAT
TACTTCTTGAAGAATATCCTTTTCAGTTAATAATTCATTAAACTCTTGTTTGCTAATTGT
TGCTGCTTCAATTGTATCATAATCAAATACATCTCCAGCTCTTATTTCATAATCCCAATT
ACCAATCTGATACTGATAATCTGCTTTTGATAATTCACGTAAATTAGCTAAGTATTTTTC
TTTGTCAATATATGGATTATGCCAGAAATTCATTTCAAAAAAAGGGTATTGTCCTTTAAC
AAATTTTTCATTTAAGTATGTTGATCCATCTGCATCAGCAGGATTACTAATATAGTAAAT
TGCTAATGGGAAAGTCATTAACTTATCAGTTCCTCTGAGACTACGATTCAGGAATTGCAG
GTTTACTTTTTCAAATTCTGAAGCTTCATCAACAATGATTTTATGATAAGCACGACTTTT
GAATTTCTCTTTGTCTTTTTCCAGAAGCATATATGAATAATAAATCCGTGCATCATTTTC
ATGGTTAATGAAACATCTTTTACTTTGATTGTGTTCTATGTAATCGAATGGTTCTGTCCA
ATGGTCCAGGTAATCTACTATTCCTCCAGTCGCTATTACATTATCATAAGTAGACCGTAG
AATTAAGCAGCGGTAGTATGGTACTTCATAATGTTGTAAGGCTAATACTGCTCCAAGCAT
ACTTTTACCAGAATATGCGGAACCTCCAATTAGTTTTCTTGTGTGGCGGTCTGCAATTGC
ATATAATTGTCGGTCGTATGGTGTTACTGGAATGTATGGGTTTTCAAGTATTGTTCTTCT
AATCAACTTTTTTTGTTGTTGATCTAAGTGTATTTTCTTGTAATCTACTCTCATAATTCA
TCAGTGAATTTTTCTAAATCATCGTTAATGGTTAGTAATTCTTCTAATTCTTTGTCGTTC
ATATGGATTACATTATCATTTTCTTTGGTGGATGTTTCAATACTTCCTTGTATTTGAGTA
TTATTATCTTTAATATCTGTTGGTTGACCATGAGCTAGTCTGAAATTTCTATAGATTATT
TCCGCAGATTTATTTAGATTTAGAAAACTATTTGCTCGTGTAGTTGGAGCATTCTCATTA
CTATTTTCTTTTAGTTCTTTTTGTATTTCTTTTAGTAATTCAAAATCATCATTGAAGAAT
TCTTGAAACTTAGCATTTCCTTGTTTGAATAATTCATAGTTCCACTCTCTTTCTTTTTGA
TCCATATGATTATCATATAATCTGCATCTTTCCACCCAATTCCATCTTGAAGAGAGTGTT
TTTAGTTGACTTAGTGAAGGTATGTTTTTTTCTTCAGTATGCTTCGATTTCTTCAATTTT
ATTATCTGTGGGAATGTTCTTTTGGAACCTAAATCTCTGTATTCTTTGAATAGTGAATAG
TATTTTTTTTGAACATTATTGTAATGTTTATTCTTTTTTGTTGAACACGAAATTTTAAAA
TACGGGATTTTTTATTTTCATAGTGAACAATATAATATTGTTTAGTTTTGAATTTTGAAC
AAAAAAAAGTTTTATTAGTTTTTTATAATAGTTTTAGGAAATTCACAATAACAAACTGAA
ATCCATCAATCAAAAATACAATCAAAGCACCAACAATTGCTATGAATACTCCACTTTTTA
TTGTGAATAATCGATTATCATCATTAGCTTGTTCTTCCTGAATAGCCAACTGTTTTGTTT
GAATTTCAATACTTTTATCCATTTTCTTTATCAGTTTTTCTAATTGCTTATTTTGAAACT
GATCACTTGCTTCCAGTTTACTTATACGAGATTCCTGTTTACAATATTTCTCATGTAAAT
CTTGAACTTGATTATGGGTCATTATTGTTTCCTCCATTGTTAGTGGAGGTGTAAGTTCTT
GAAATCCATCCAACTATCCCCCCTAATGCTACTGCTGCTAGTTCGTTGTTTCCAATATAG
GTGCTTAGAATTCCTATAATTATTATTCCAATTATTGCTAATGTTGTGTTGTTGAAGTTA
GTCATTATTATTTTCACTCCTAAAAAAAATTTGTTTTTAAAAAAAACATTGGGGAAGGGA
TTTGAACCCCTGCGATACTAGTGTATCATTAGATTAGCAGTCTAACGCCCTACCAGGCTA
GGCTACCCCAACATTGTTGGTGGAGGGAATTGCACCCTCAACTTTTGAGTGGTTACTAAG
TTACAATAAAAAATAAGCTATTACCAACATCATGAACATGTATTAAAGGAGATAAATATT
TATATTATATTTATGGGAAGAATATTTTTTTTTAAGATATGTGATATTAATTATTTAATC
TTTGGAGGATACCTATATATTTTAATGAAGAAAAAAATCCAATCATTCTATTATTCATTA
AAAATTTGCTACCATTCCCATAATAATTTTTAAGAATAATAATAAATGAAAAGCGAATAA
TAAACGGATAAGAATATTATTTTTAAATTGTCAAATAATGATAATGCGAAGCTAATTGTA
CAATAACATATGATAAAATATTTTTTATAAGTGTTTTATTACAAGACCACATTTTTTACA
AACATACTCATCATGCCACTCATCATAGTAAACCTTTTTGCAATCTTTTTTCAGTTTTTC
ACAAACAGGACACTCTATTTCTGCATGTTTCATTTTTTAAGGGAAATACACACCCGCAGT
TTGTGCAATTATATTCTGCACGAGATTCATCCAAAACAGTTAAAGGGGATTCAGGAAACT
TAATATAACAAGATGTCTTATGATTTCTATAATGTTTTTCTTGTTTTTTCCTTTGTTTCC
ACTTCCAGTTACAAACAGGACACTCTATTTCTGCATGTTTCATGTTTTTAAAAAATGTAT
TAGCATCCAAATAATATTATCCTCCCATATATAATTATAATAATGTCTTTTATTTTTTTT
TCATAAAAAAAGATAAAATTATAAAATATTTTATCCCCTTCAATATATAGGAGTGGAAAA
ATAGAAAAAAAGATTAAAAATAAAAAAAATTTAATCACTTAATAAATAACTATCAAGAAA
ATGCTCACCACGACTGATATGTCCCTTACTTACAATCCATTTAATCATGGGATCCATCAA
ACCTTTTTCATATTCTTTAATAAAATAATTCAATGTCCAAAAAGTAACCTTTTCATTAAA
AATTATCCTTTCAAAAAGTTCTTTAGATGTCTTTTTAAATTTTTTTCTATGAAAAATAAT
ACTGTCAATTTCATCAATAGTTAAATACAATGCAATATCTTCACCAGAAATATAAATACA
TTCCTTATCAACATCATACCAATAATACTTCTCATTAGGATTTCTTAATAATTTAAGATA
CTTGATAAAATTCATATTTTTACTCCTATTCTTTTTAATTCTCTTTTCACAGTTCTTTCC
TCCAAACCAAAATCACTCAAAGCATGCTGTGAAAGAAATGTTGTACCAGGCTCCCTTAAC
TCATTACTAATTAACTCCCCATCATTAATTAATTTCCTACGTTTTTGTTGATACTTTGCT
TTCTGCTCACGTACCGCCCAGGTCCTGCAAGCTTCCCTGCAATAACCTGTTTTATTCTCA
AACTTAATAAATACTCTGCCACAGTATTTGCATTTGCTGATGTAGAATCTTGTTTGTGGA
TCATTTAATGTTTTCTTCAATAACATCCTATCATCTTTCTAAATTTTTAGATAGTAAGTT
TTTCTTCACTTCAAAAACAAACATGTGAAAAGGTAAAAGTTTACCCTGATATTTCACATA
TTCATTAATTACTTGTGATTTTTTTAATGCTTCAGTTATAGTTTCTTGTTTTAATTCTTC
ATTTATGCATTCAAAATTGGAGACTGTTCTACTTAAACTAATGTATACTCTTTCATATAG
ATTAAGGTTATTGTTTTTCACATTCTCCACCACAACAAGAAACTGAATTTTCAGAAGTGT
TGAATTTACCATCTTCAATCATTTCAATCAACTCATTAAGAATACGAGTAACATGCATCA
AAGTATGAAGATTAATACTATGAATTATAACATCATCATACTCCATGTCTCTCAAACTAC
CTGTGCATTCTTTATGAATAGTAAAAGATTGTTCTTCAAATTCCCTGCTTCTTGTTTTAA
TAAAATATAGTAATAATTTCTCATCAATCATACATATTTATCCTCCATGTATTTTTTAAC
TTCTTTCATAAAAACTCCTGATTACAATATAACCTATCTTAATCATCAAAAAAATCAGTA
AGACATAACACTAAACAACAACTAATAATTACACTTACACTAAAAACTACCCAACCATCC
ATCATACTATATTCTCCCCCCATTATCTCCAACTTGTTATTAACTCATACTCCTCATGAG
TTAACTTCTGTTTTAACCTTTCATCAAAACTTCTTACATCTCCAGGATTATTCTCATCCC
AACAAGACTCAATCTTGGCAGTTAATTCATTAATTTCTTCCTTATTCATTTTCAATATCC
TCCTATTTAATTTTGAAAGGTTGTAATGGTAGTGTTGGTCTTTCAAATATAATCTGAAAC
AAAACAGTATCCCCACATATATCTGGATAAATACTTTTTAACTCATGTTTCAACAAATCA
ACATGCCTATAACCTTCAAACCAAGCATGATTATGTTTCAAATCTTTAAATTTAACCTGT
TCAACTCTCCGTACTACCCGAAGCAGACAAAAACCCGTCCCCTCAAACCAACATTCTACA
AGGTCACCTTTTTTCAATCCTTTATCAGATTTTCTTATTGTAGACATTTTTTTCCTACTT
CTTAAAGGTTCAAAGAAATACGGTTTAAACTTCAATGTTTTCATTTTTATCAACTTCCTT
TTTTACATATCTTTGCTTCTTTTTTATCATCATCCAACACTATATTATGTTCTTTTAGGT
ATGATTTTCCAAATAATTCTTCAGCATGTTTACATAACAGATAAGAATCAATTACTCCTT
CAGCAGATATTATTCCTTCTTTTTCTAATTTTTCAAGTATATCATTACAACTCATTTTTG
GATCTCCTTTTTATTCCGGACTGGTTGTCTTTGACTTGGTTCAGGAACACCACACCTCAT
ACGTACCTTTTCAGCAAACTTAACAAACTCATCTGCAAATAAACTTATTCTAAACCACAT
CAACTGCCACCTTAATATAATATGTTCCTCATCTTCTTTCTTCATGATTCCACCTTTTTT
AACCTAGTTTGGAAGTTATTTCCCCCATAACGCCTAACATGATCTTCACAAATGATTCTT
TTCACAAAGGAATCTTTTTGATGTCGAGTTAATCCATACCTTTTCTGCAACTCATCAACA
GAGATATTTAATTCATGTTTATAATCTCTCCTGAATTTCAAATATGTTTCTTCACCTTTA
GAAACATCATAATCCGTTTCAACAATATTAAAAACTGGTTTATTCATTTTTTATCTCTCC
TTAAAAAAATGTAAACATAAATGTTTACAATTTAAAAAATATTTATAGTGAATTACGTAC
GTATACGTACGTACGTATGTGCACGTACGTGGTTTCCTACAGGTTCTTCGGAAAAATGTA
AACATAAATGTTTACAAATCATTTTATCACCAAACCATAATTCCGAACATCATAACCCAA
ATCATACACATGATCCAATAATGACTTATACGGAACTTCTTGCTGCTTACTAATATTCTT
CAGCTGATCTTTACCAATAAAACCAGCATTATCAACAATCCTATTAACTGTACCCATCAC
CCCATCAAAGACATTTACACTTCTTTCATGTTCTAAACGCTCAGCACGAATCTTACATAA
TCTTGATTCACGAGCATTAATCTCATTTTGCAACTCAGCTATCTCTTTTCTCAAACTATT
TTCCTCATCTTCAGATAAATCAATAGCTAATCTTAACTGCTCTTCTAAAAACTCACTTCT
GGTTGTCCCCAGTTTGTTTTTAGCTTCCCTCCATACTTCAGAATCAACCATGAATGAAAC
CTGTATTTTTTCACTCATACATCATCACCTAACTCTTTCATGACAGTTTCTAATTGCATT
TCCTTTGCAATTAAATCTAATTTTAAAGACCTGATTTCCTCTTTTAACTGGTCCTTTTCA
ATTAACCTTTGATTTTGTTTACTCACATACATATCAATGCAATGTTCCACGCAGAAACGT
ACAGTTAAACCATACTTTTCCATTAACTTTTTTGCCCTTGTAGTAATCCTACCAGATACC
ACTTCAGGGAAATTATCCTGAGTATTCATACTCTTACACCTAACATTTTTTCTGCTTTTT
TAATATGTTTGCATTTATAATGTTCTACTTTTTGATGTCGGTAGAAATAATCTTCACAAC
TACACCACCATCCATCTATACTGTCCCAGTTCACAGTATTGGATCCAGTATTTCCTGATG
CCTTGAATTGTATGAATATTATTTCTACCTTAGGTTCCATAATTAAACAGCAACCTCTTT
TTCTTGATTTTTAAACCAGTTATATGCTTCTTTGTTTTCAGCTTTGGTCAGTTCACCATC
TTTGAATAACTTCATTCTATGAGTATTAATCATAGATGGAGTTATTTCCAGTGAACTGTC
CTTTTTATGAATATACTCGCGGATTTTCTGCACAGCACCAGTAGCCTCAACTTTCTCAGT
TCTTGCAGAACTACCCTTAGCAGTTTTAACATTAATTTTATCAGAATCCACAACATCTTT
TTCAACAATTAGAAACATGTTTGCTAATAAATATCTTTTTAAGTAAGTAATGTAACTGCC
TTCACTTTGCATTACATTCATACCTTTATTTAAAGCAACTATTTCAGGCATAGGAACACT
GGTGATGACAGATTCCCCAGGTTCATTCCAATTTCTGATTTTCAATTGAGCCAATTCATT
TGTAAATGAAAACTCAATGAATAACTCCTGTTCATAACATTCACAAAAGATTGCAGGCAA
AATATCTTCTAATTCATAGTACTTAAATTTCCCAAATTTATTGTACCCACTTTTAGGTAA
TTTTTTATGCAGGAGATTCTTTTGAATTCCTGCAATTTTCTGATATATAGTCATAAAAAA
ATAATCCCCCTTTTTACATATTCATCGACTTTTTCATCAAATCCCTAATTTCTTTTAACT
GTTCATTTTCACTTATTTTTCTAAACATGGTGTTCAGAATATATTCATCGTAATCTATAT
TTTTTTCAGCACATAACTCTTCCATCAGTGCTTTAACTATTCTAACTTGATTTTTCTGTA
CAATATGTTTAAAGATCATATCAAAAAATGAATTCAATTCTGCAGGATTTTTAGCATCTA
CATTATGGTCTAAAAAGTGGATTGCATTAGTTAATTTCTCCTTTAACTCTTTTTTGTTTA
TGATTTCTTCAGTAAATAGTTCTTTCTCCATTAAAATTCCTCCTCAATAATCTCCAATAC
TTTGTTTTCATCAATATTTTCCAAAGATGCTTGAGAGTGTGGAATGAAACTTTTTGACAC
CCAGTAGTCATGATACTGGATTTCACCACCACACAGTTCCTGTAAAGTATTCATAATATC
ACGTGTGTTCTCATAAGTACTGTCATGAAAAACAATTACAGATAAGAAAATACCATGTAA
GTTTCCATGAGCAGAAATACGTCCGCCCATTTTTTCAACAATTCCTAATAATTGATTGTA
AAACCTGTTTTGCTGATCATAAGTAATCATACTAAACACCCATTATTATAAGGATCCATA
GGCCCTACTACAAGGAATGCAAAAATGAATAATAAAATTATTAAAATTATTCCTTTTGCA
AAGATTATTTCAACTTCATACTTTTCTCTCCAGGACACTTTTTTATGAAGTCTAACTGGA
GTAGGTTTATTGTTGAAGAGACTCATAATTGAGCCTCCAACACTTTATACTTAACTTCTT
TTTTCATACCAGGAGTATTAAGGAAATCTTTCAATTCCCTTAATCTGGTTTGTTCTTCAA
CATACTCTGCATACTCCCCGTATAATGTTTCGAACAATTCTCTTTGTTCAGGATATTCCT
GTGCTAGAAATACGAACAGTTCATCTATTTCACAGTAATCTTTGTACTGTGACTGGTTTA
TACGATATAACGCATTACCAATCTTGTTTTCATTTTTCCAAAATTCAGGAGTGAACATTC
ATTTACCCCCGTAAGCTAGTTCACCTGCACGAGCAAACTCCAACATTTGATCATCAGACA
ATTCATAGATTTGAAATTGCTTATATTCATCAGCCAACCTATGATCTATTTCTTCCTGTG
TTTCATAATCCTCAGACAATAATATCTGTTCAAGATAATCAAACTCGACCTCATCTTCAG
GTGGAGTAATCGTATACATTGAGTTGTTGTATCTGCAACTCATACGCCCACACTCCTGAA
TTTTTGGAGTTCTTCTTCATAGACTCTGTTTTGAGCCTGAAGCCTTTTTGCTTTTAAAAA
TACATGTTCAATTTCTTCTTTGGAAGTTAACCTTCCAAAAACTTCAATCTTATTATTATC
CATATTACCAACTCGTGGATTTTTTAGAAGATCTTCTTCAGAGTTGCCGCTCTGATTTTT
TCTTCTTCAAATAACTATTATACTTAATACTATATAAAGGTTATTATATTTACCTTTATT
AAAGGTAAATATATATACTGCAAAACAGAATATATTATATAAGAAATATAAAAATGGAGG
TATGCCTATTTTAGAATATACAACAAAATTAAGTAAAGGCGGACCTAATTCTATTAGGTC
AATAGTCCCCCAAGATGTGATAAAATTATTAGAATTAGAACTGGGGGATTCATTACATTG
GATTGTAAATATTGATGAAGGAATAACTGTATCTATTGAAAAAGCTGAAAAATAATCAGT
TAATCCTTCACAAATATTTTTAGTAAATATTAAATATCTTTTTTTATAATAAATAGTATT
GGTTACTAAAATTTTTATCAACTCGTGGTATCTATTTTTTGTAGCCTCTAGTTTTTGAGA
GGCGAGATGTTGCCGCATCACAGACCCCCTCATCAACTAGGAATTACATTTACTTATTTT
GCATTTAATTGTTCTTTTTCAACCTTTTTTTGTTCTGATAACATCATAAAATATGTAAAG
TATAATCTATTTACAAAACCATATTCCCCACTATTTTCTCTCCCAATAGACTCTATAATC
CCAAGGTCTTTAGCTCTCTTTAAAAAATCATTAAAAGCTCTCTTCTCCCCATTTGTCAAA
ATATTTTTCACATCTGATTTTTTAAAATCCATTAATCTATTTTCCCCTAATTTAAGAAAA
ATATCAATATATGTATCGCTTCTAATTTTATTTAATTTGGATTTTAATTGTTTTTTAGCT
AATTCATCTGTTGCATCAGTAATACCAGAAAATACTATAGATTCATTTATTTTTAAATTA
TCTTGAGCATTCCAAAAAATTGAATCACCAATTTGTTGCATTATTAAAGGCATACCCCAT
GAAAAATAAACCATAGGTTCTATAGAATTATATCCATCTTCAAACTCAATGCCTACATTA
TTAAATGAAGTTGTGAAGAAATCTTCAATATCATTATCCTCTAAATTATCAATTTCAATT
AAATTAAACATTCTAGAAAATGATTCATTAATTGAACATAATTTTTCAAATTCATGAGGG
TAACTTATTAATGTGAAAACAACAGGCATATGATATTCATTAACCAATAATGTTTCAGAT
AATCCTTTATACCAATCTGTAAACTCTTCATTATCAGATAAACCATTAAGATCATCAACA
ACAATAAATATTCCATAATCTTCAGGTAATTCTTTACAAGTAGTAATCAAAAAATCTGCA
AAATGTTTTTTAATATTATGAATTAAATTAGTATCATTATCTTTTAAAGATACTCCAGTC
CCTGCAACTTTAAATTCATTAATATTATTCAAAATTTTATCAATTACACTTTTACCTAAA
TAATCCTTTTTAAATTCTTTTAATAATCCATCAAGTAACTTTTGTATTAATTCATCAATA
GTTTTTCCACCACCATTGTTAATATGGATTGGAATCATTTGAAAATCATCTTCTGCTTTT
CTTGAAACATAATTTATAAATGATGTTTTACCCATTCCTCTCTTTCCAGTTATGAAAAAA
TGTTCGGGAGCTCCTTGATTTTTAACTTTAGGTAAATATTTTATAATTTTGTTGATATCT
TCTTTTCTTCCTTTAAAATTATCTGGAGATACTGGTCTCCCTGGTTCAAAAGGACTTTCT
GGGAAATCTTGTGACATGATATTTTTACCCCTCAATACTCATTTATTACAATTTACATTT
TGTATTTAACTGTCTTATCAAATTTTTATTACTGTATCAGCTAGAAAATCTGGTGTAACA
TTCCGTTTCTTTTTTTGATTTAAAATAGTATTGGACCATTTACTGAACTGTGTAGCTTCT
TTACTATTGACGCGATAGCCTACGGAAATCATAGCATCTAAATTATAGAGTATGGGTTGA
GTATTTGCTTCAGGATTAATTTTAATAACGATTCCACTAATAGTGGAATCGTTTGGATTA
ACAGTTACTTCATCTTTAACAAGTTCTCCACTATCAAAGATATTATTTAAATGTTTACTA
ATGTTTTTTGGTGTTTTATTAAATAAATTAGCCATTGTTTTTTGTGTTGCCCATAATGTT
TCGTTTTCTTGATCAATGATAACTTTAATTGATACTGGCCCATCTTCACTTTTATATAAA
AATGTTTCAATAAGATTACTTTCCACCATGATTTATATAACCTCTACAATTTTACTCTTC
AAAAAAGTTTTTTTATTCTTGTTTTTATATTTATGTTATAAGTTAATATAAATGTTTCTA
TTCAAAAAATAAACCTTAAAAATACAGTATATTCAATGAAAATAATGAAACACTAAACAT
TATTCAGTATGAAAAAATGAAACGCATTGTGTTCTAAATTTTGGTGTTTAATTAGTTTTG
TGAACTGTTAGGTTATTGTTCATTTTTATAATGAATATTTTTTATCTTTTTTTGTTATTA
AAATGTATTGATATTTTTAATTAAAAAAATAGAAACCTTTATATACTATGTAGTACAATA
TAATAATAGAAATACATGGAGGTGAAAAGTTGTCAGAATCAGAGGCAATAAAAATCATAA
TATTGTCTTTGAGTATAATCCTAGAAATACTCAAAAGACAAAAACACAAGTAGGAGGTTA
ACCCCCTCTTACAATTATTATATTTTTTATTGCTTCTTATATAAATTTTTCTTAAAAAGG
AGGTGAAAAACAAAATGACAATAACAAGCATCATAATACTAATATTATTAATCATATTAG
CAATATTAATCTACATTAATAGAAACCAAAAATATACTTGGATAAGTTTAATAATATGGT
TGGTTATTTTAATAAGTTTCATCTACGAAACATTAATCTAACCCCCCCATATTTTTTTTA
TATTATAAGCCAAAAAAAAGGAAGATGATAAAAAATGACTGACTACACCAGCATAAGAAT
AAAAAAAGAAATAGCTGAAAAAATACAATTAATAAAAATACAAAACAACTGCAAATCACT
AAACGAAACACTAGAACAACTAATACCCCGAACAGTAAACGAAAACTACGAGTTCATAAA
AGAACAACCAATATTCACAATAAACAACAAACCCATAACATTCACAGACCTGAAAAACAA
CAATACAGGCAAAACATGGGGAAATGAAAAACAAAACGCAACAATAGTATTTAAAGATAA
ACAAGGAGCATTTATAAGATTCAATGATGAAGATGAAGTATTCTTAGAATACTACCACTT
CATCTAAAACCATTCTTTTTTTAATATTAAAAATAAATCCATAAAACCTCAAATTTTTAA
AGTTTTAAAAATTTGTTCCATAAGGTACATAGCATCATCCAATTCACCTTCAGAGATATT
ATAAAAAGGATCATAATCTGCTTTTTTTCTTAAATTAAATAAATCATATAGTTTATTCCT
AACAGAATACTGATTGACCTCAAGAAGTTCATATTGAACTTGCCCATGTTCTGTAATAAA
CTCATTTTCACCAAAGTCTTCTTTAGACAATGGTTTAAATTTATAAACTTCCTGAAGCCA
CAAACTAACATATAAATAAGCACTGTAATAGCACCTATTGATAATAGTACTAAACACACA
ACGATTATCGCAATTTTCAGGGATTAATTCTTCTTTATTTTCATATAACTGCTTAGTGAA
TTTATATAATTCATATTGAGGGTATTTTTCAATATTAATGTATAAAACCCCCAATAAATT
ATTTAATTAAAATATAGGTATCTTTATAGAAATCAAACATATTATTTTCCTTTGAAAAGA
TAACCATATCATTAATAATTTGGTCTAACAGAGAATATTTTTCTTTCCTATCAATATGAA
ATGGAACATTAATTGTAAATACTTTTTCAGGATATGATCCATCATACACTGTATCAATAG
ATATATTTTTTCCTAATCCTAAATTGTCAGAAATTTTTATAAGTTCATCATAAAATTCAG
TGATTTTACTTTCAAAAGACTCTGTGGATGATTTTTTAAAAAACTTATTTTTAAAAAAAT
CCCCTACAATATTAGAAGGTTTATTATATCCATAATTTTTATCAACTGAATACACCATAG
ACACCACCTATAAATGTTTATTCTAAAGTTTTTCTAATAATATCCAATGTTTCAGGATTA
TTTTGAACATCAATAAGAACTGTATTACTTTTTATTTCTTCATCATTCCTATAAGATAAA
GTAAGAATATCCTTATTTATTTCCCCATCTTTTAAAACAAAAGCTGCATTTATAGGTATA
ACTTCAAGATTTTCAAAGTTTTCTTCAAGTTTTTTTAAAAAATCAACATTAAAAACTTCT
TTAAGGGAAATCCCCTCTTGAAGCACTCTTAAGCTTCTTGCATCCTTTTTCATTTAACTT
CCTCCAAAATAGTTATTTAATAAATATCTTTTATTAAACATATAAACCTTGTCAAAAAAT
TTTTAAATTAATAAATCTTCAAAGTCATTTTCTTTGAAATTTTCTTCTTTTTTCTCAATA
TTTCGTAATCTTTCATCAATATCATAGATTAATTTTTCATAATCTTTGATTTTTTCATCT
TTTTCAACAATTGTATTTTCTAATTTTAAAAATTCTGGAGATTTAACAGTGATCTTTTCA
ACTTCTTTACTAATTGATAATGCTGGTAAATGTTGAATATATTCCTGTTTTAAATCAGCA
GGATTGGTCATGAAATATACTTCATCAGTACTGTTTTTTGATTTGCCTTGCAGGTCATTT
ACTTTGTCCAGACTCATACCATCATTATATAGTGTTGAGGCGTGGAATTTTCTGAGCATG
TGGCTGCGGAATCTGTTGTAGTTTCCTGCTTTTCCTAATCCTAATTCATTGTTTATTTTA
ATGAATTGTTGGTTTAGGTAGTCTTCATGTATTTTGAATAGTTGGGATTCTGGTGTTAAA
TTTTGTCTTGATAGTAAGTGATGATTTATAGCTGTGACTGCTTCTGGACTGCAATAGGTA
ATATAATATTTCTGTGTTTTTTGCCTTAAAATATTAAAAGTAGGGACAACATTATCAATA
TTATTTAACACATCTATCATTTCCATAATATTATCTGTATTATGGTATTCTTTAGTTGCG
TTCATATAGTCCATTACAGTTAAATTTAATGTTTCTCTTCTGGCACAACCTGAACTACTC
ATGAATAATATAATAGCTTTCATGGTAGAAGTACATATATTAACTGCTTCACGAATAATT
TCTTTATCAGGTAAATCTTTAAAACTGATCGGTTTGGGATTATTATAACTTTTTTTATCA
ATTCGTGGCAGATCATGTATTTCAATTTCAAAATATTTATATACAACAAGAACTGGTGTG
AAAGTATTTGATACAGTGTTATAATAATAATTATCCATCAAATACTTGCGAAAATTTAAT
AATCTTCTTTTTAATGTACGATGTTTCCATCGTATACCCTGTTCTTCTTCTTTTTCTGCT
TCTTCGATTAATTCGGCTAATGATAACTTATTCAATTCACAATATTTATTTATTGAATGT
TTGTAAATATGCTGTGTTGTTTTTTTATGATTTTTAACAGAATGGATCTCATTTAGGATT
TCTTCATTGGTTCTCATATTTATCAAATCTATTTTTCTCTTTTTATATTGTTTGATGTAG
AGTGCATTTGATAAGTATGTGATAATATGTCTACTGTCGTGTCTACAACGAGTTAATTAT
AACATACTTAAAACATTCTAATCTATTAAAATATTATATCAAAAATAGGTTTTAAATTTT
ATCCCCCAAAATTAAACCCTTATCCTCGTACTGCGTTTATAAAAAATGGAAGTAATTAAG
ATAAATTTCTCCGTATTCATCTTCAAATCTGATTAATGCTCCAAGGTTATCTTTATAGAT
TAATGTTGCTTTTTCCCCATTACTCCATTGTGTGCCTACTTCCGATTGTTTTAATTCATT
CCATGATACATTTAAAACTGTTTTTTTGTTTATTAGTGTGAATGCTGGTTGTTCTTGTAT
GTATTCTTCAGGTGTTGATGTTCCATCAGGTAATAAATGTTTTACTGTTTCATTAACTGA
TTTGTATCCGTTGATTTCCTGTATTTCTTTTATCTTGTTAATTAAATTTACTTCAAGTTT
TATAGTACTTCTTTCCATTATTTAATTCCTCCTTTAAAAAAATAATAAAAAAATTATAGT
CTTTATATTAAATGGATTATAATGATGACTATAATATTTATTATAAGCATTATTGCGTTT
CGTGTCCTGTTTTCTTCATTTTTCATTGAATATAATAATACTACTGCAAGTATTATTAAA
ATTATTTGAATTAATGTTAATACTGTGGTTATCATAATTCCCCATCCCTTTTATTTAAAT
ATTAGTAATGACATATAATTTATTATAAATTATTTTAAAGAGGGATTAATTTTAATCCCC
CATTAGGGATAATATTATTGATATTATCCCCATAACCATATTTATTAGAAAATATATGTC
ATTTTTATCAATGTTTTTCACCTCCTTGTAGTTCTATTATTATATTGGTTACTATACTAT
ATAAAGGTTTCTATTTTTTACTACTTATTTTTATAAAAAAATAAAAGAATTGTTGATAAA
ACAACAATCTTATTGATACAAATCTGAGAAAATCCAAGCTGGATCATACGCTAAAATCGT
ACCATTCATACACCAATTACTTGAAACACTGTTTCCATCTAAAACTGCTGCAGGGTCACG
ATAAATCCAAGAACCCCCAGTATACTTAGAATGTTTCAATCTGAGCCTTACATGCCCAGT
CCCACTTCTACATCGTACATGAATGAATTGTACATTATAACCTAATGCAAGTCCTAAACG
GTAAAATAATTGACTTGAATCAGTACAATTAACTCCTTGCTTATTATATATCTTATTAAT
AGTTTCCTGAGTATTATATTTGGAGTTATAATAATATCCATATCCTCTTCCTCGGATTTT
GTTCAAGCAAGAGTCTATATCTGTTACACCACCAAATTTGTCGGTGAACCTTTTTAATGT
GATGTCTGTGGTGTCGCTGGTTGTTCCGTTGCCATGTGGGTCTTCAAGATAAACAATAGC
TGGACTAACACCATTTAATACTTCATAAGCTGATACACGATTAGCCATACTAACATAACA
ATTCTTATAATACTTTGTTCCATCAGTATCAGACACATAATTTGGGAGGAAACTTAAATC
TTTATTATCAAATATTGCACATTCAACAAACTCTGCAATAAGACTGTCTTGTTGATAATT
CGTACCTCTCTCTTTAATTGCTTTTATAATTGTGCCATAATCAGCTACACGATTCATGTA
ACTATACACTTGTCTTGTATTAAACTTTTTATTATTATTCACTAACCAATAAGCAATTCT
ATTCATCAAATAAGTGTATTTTGTAAAACTAATATGTTCATTAACCATTTATTTTCATCT
CCTTTTTAAATTTTAAATCATCTAAATATTCCTTTAAAATACTGCATTTTATTATTGCAT
CTTCAAAACAACAAGGAACCTCTTTATCACAAGGATCATAAAATGCACATTTTTCACATT
CTTCACTAATCATTCCAAAATAATCCTCCTATCAATTTTTAATGTACAATCAGTAATCTT
GTTTGTTCTAAACGAGGATTCTCTCCAGCAGCTCCTCTTTTTACAATACCTTCCAACATA
TAACTTCCAGGGTCTAATCTAATCGGCAGTATTGCTCTACCATTAGCATCAGTTGTTCTT
TCATAAGAAACACCATTTACAATAAAATTAATTACCACATTTTCCCCTGTGTTAATTTCA
GGATTGGAAGAAATAATTTTACCGTAAACAGAATTTCCATAATTTAATTCTAGTTTAACT
CCATCTTCTCCAGCAAGGTCTGATAAGCGACCCATTACTAATCTAGTGTTTTTTAAATCA
TCCATTGTGGTTTCTAATATTTCTTGTCTTGCAAATGTTTCAGAATGTTCATCATCCACC
AAATCTGCATTTAAACCTGAACCTGCTCCATCCACTTCTTTTAATTTATTTAAAATAGTG
GTGGGTGTTTCAATGTTTCCATCTTTACCATCTTTTCCTGCAGGTCCAGTATCACCTTTA
CTTCCTTTATCACCTTTAGGACCTGTTGGTCCTGTTGGTCCAGTATTGCCTTTAGGGCCT
TTGAAATTCCCCAGTAATATTTTTTTAACCATTAAAAAAAACCTCTTCATTTATTTAATT
ATACTCCTATTTTCATTTTCACACCAGACACATTATAACTCATACTTGTACCGCTACTCC
ATGCACCTGCTTTTGTAATTAATCTCCACCTATTAGGATTAATTTCTGCTCCTCGACTCT
TATTGGCTGTGTTAATTGTTTGAGAATCATCACATAATACATAACTTAACTTATCATGTC
TTACTTTGAAAATTAAACCAAAATATTTTCCTGCAACATTCCCATATGTGCCCTTTTGGT
CGTAAGTTCTATTTGTTCCACCATCACAAACTATATCAACCCATGTACGGCCTTGCTGAG
GGGTGCATACTACACTTATTTTTTCGTTTGTTTCTGGGTTCAATAAGATAAATTGTATAT
TAGAGTTAGTTCCTAAGCTGTTAGGACTCCATGATAAAAATAAGTCAAAGTTTTCAAGAT
TAAAAGTATCTGTACGATACCCTTCTTGAGTGCTCTGCTTGTAGGGTATTTGTATGGTAT
CGTGAACATGTGAGTGAGTAATACTTTTATCTGCAATACTGCTTCCTTCAGGCAATATAA
CATCAGATAAAAAGAAACTAAAATCTCTTGTATTAATATTGGTTTTACCATCCAAGCTTG
ATTGAAGATCAGTAATATTGCTGATACTGTGACTGTGACTATTGTTAGCTTTACCGTTCA
ACGCATTAGTCACGATTTTATTCTGTACAGGATTAATAGAAGAACTACTTAATGATGAAT
CAACAACTGTTTTATTAGCACCATCATCAATATTTTCCAATTTTTTGTCATGATCATAAA
CAAAACCATTTACACTTGACTGTCTATTAATTCCATATTCATACACCCACATGCCATCAT
CACTATCCTGATCAAGGTCTATACTATCACTTGTATGGCTGTGGTTAACATTTGCTTTAT
TTGCTAATTGAGTATTCATTTCTGTTTGTGTAACATACCCTGATAAGTCAACTGTTGTTT
CAGTTGAACCAATTTTTTCAAATTTATTATTAACCCAAATATACTCATCATAAATATTAC
CCTCAACAGAAGCTGAAGTATTTAATTTTAAATACATTACTCCAACTTCCCCTGTAGATG
GTAATGAATTTACTATTGTTGCTGTAAAACCAATTAATGAATTTATCTGAGAATTAATAT
TATTTACTTTCGCATCTATTTCAGTTTCAGTATAATACCTTTCATCATGATTATGTCCAA
CATTTGATTTTCCACCTAATTTTGAATCAATTTCTGTTTTCTTATAATAATCATTTAAAT
GTTTAACAGCAATATAATTGCCAACATGTACCTGATAACTATTTGCTCCATCTTCACTAG
CTATATTTAAATATAAATCACCAGTAGTTTCATCATGTTCAAATATTGGAGGGTTGGTTT
CATCATTACAATAACACCATAGATTTCCTTCAGCATCTCCTGCTAAAGTGAAAAAACCTG
GTGGAGGTACTGTAATATTTAATAATGGGTTTGCCCATTTGTCGAACAATTCATCAATGG
CTTTACTTACTGTATTGGCATCTGTCTTTAATATGTCGATGTCTAGGAAATCTTCAATGT
TTCCTATTTTTGTGCCTAGGTTTGTTTCTCTTAATCTTGCACATGCATTATTTAATGCTT
TTCTTTCATTTTCAGTTAGTTTAGTCATATTACACTCTCTCGTTTAACATTACCTATTTT
AATCTCTTTGTCACCATTTAGATAAGGATTATAACTTATTTCTGTTATTTGTGCTTTGAA
CACCTCTGAATCTGGGAATCTTACATATAC

In some embodiments, the compositions further comprise at least one additional virus capable of inhibiting the production of methane in vivo in the gastrointestinal tract of a human. Similarly, in some embodiments, the methods in which the viruses and compositions are used comprise the administration of at least one additional virus capable of inhibiting the production of methane in vivo in the gastrointestinal tract of a human. In some embodiments, the at least one additional virus is a virus capable of inhibiting the production of methane in vivo in the gastrointestinal tract of a human selected from those disclosed herein. The viruses may be stabilized as discussed above.

In some embodiments, the compositions further comprise at least one additional active substance which alleviates the symptoms of irritable bowel syndrome. Similarly, in some embodiments, the methods in which the viruses and compositions are used comprise the administration of at least one additional active substance which alleviates the symptoms of irritable bowel syndrome. In some embodiments, the additional active substance is an antispasmodic. In some embodiments, the antispasmodic comprises hyoscine or a pharmaceutically acceptable salt thereof.

In some embodiments, the compositions further comprise a clay, for example, Fuller's Earth. In some embodiments, the compositions further comprise silica gel. In some embodiments, the compositions further comprise fibre. In some embodiments, the compositions further comprise probiotics. In some embodiments, the compositions comprise one or more of a clay, silica gel, fibre, and probiotics.

In some embodiments, the compositions are configured to provide location-specific release of the virus to the human. For example, the composition may be configured to provide location-specific release of the virus when the composition enters the target release area (such as the small intestine) in a patient's gastrointestinal tract, thus allowing for targeted reduction in methane in the parts of the body where its production is most problematic.

In some embodiments, this is achieved when the composition is in the form of a tablet which is coated with an enteric coating or, more preferably, encapsulated in an enteric coating. The enteric coating protects the active ingredient (i.e., the virus) from the acidic pH environment in a patient's stomach, preventing it from being released. Once the tablet passes into a patient's intestines, the coating breaks down to release the virus in a targeted way.

In some embodiments, the composition is in the form of a dietary supplement for addition to a food or beverage. In some embodiments, the dietary supplement is in the form of a capsule, liquid, syrup, powder, food, or beverage. For example, a subject may take a capsule containing the composition as a health or nutritional composition, e.g. on a daily basis. As a further example, the virus may be incorporated into a food product such as a nutrition bar. The virus could also be incorporated into a range of foods to facilitate delivery to the recipient.

In some embodiments, the compositions disclosed herein are not solid or semi-solid compositions for administration to a ruminant, as described in PCT/EP2023/069722, U.S. Ser. No. 18/352,760, U.S. Ser. No. 18/492,384, UK 20230010863, and AR 2023P101846. In some embodiments, the methods disclosed herein are not methods for reducing methane production in a ruminant, as described in PCT/EP2023/069722, U.S. Ser. No. 18/352,760, U.S. Ser. No. 18/492,384, UK 20230010863, and AR 2023P101846.

### EXAMPLES

### Example 1

A virus with activity against Methanobrevibacter smithii (ATCC 35061) was isolated from abattoir specimens of fresh bovine rumen fluid that had been concentrated by filtration and addition of polyethylene glycol (PEG) and sodium chloride to a final concentration of 10% (w/v) and 0.5M, respectively, as described below.

First, virus particles were separated from the rumen fluid, concentrated and purified using PEG precipitation and centrifugation. Archaea cultures in 119 Broth were inoculated with the purified virus suspension and the cultures were allowed to grow for 48 hours to allow any Archaea specific viruses to initiate a cycle of infection and amplification.

Following incubation, methane production was measured using gas chromatography-mass spectrometry (GC-MS). Cultures inoculated with the virus preparation were compared to those that were not treated with virus, but were otherwise identical. The presence of active virus was confirmed by a measurable reduction in methane production in cultures exposed to the virus preparation. The viruses released during the infection cycle were recovered from the cultures by filtration with a 0.22 μm filter that allowed the separation of virus particles from the Archaea in the culture medium.

The recovered virus suspension was then used to inoculate a fresh culture of host Archaea cells as before. This process of infection, replication, extraction, and re-infection, referred to as passaging, was repeated four and six times to obtain a virus with activity against Methanobrevibacter smithii. The virus with activity against Methanobrevibacter smithii was a novel virus which had the sequence set forth in SEQ ID NO:1.

Fresh human feces obtained from three healthy donors were used in an established in vitro colon model to investigate the methane mitigation effect of the isolated archaea virus (having SEQ ID NO: 1). Because the nature of the intrinsic archaea population within each fecal donor sample was not known, a spike of Methanobrevibacter smithii (ATCC 35061) was introduced to the in vitro colon model to provide a sensitive target for the virus.

The in vitro colon model comprised 100 ml volume, acid-washed, sterilised, air-tight, glass vessels with sealed rubber lids which could be manipulated in an anaerobic chamber containing a standard anaerobic gas mixture of 80% nitrogen, 10% carbon dioxide, and 10% hydrogen. The rubber lids were self-sealing and remained air-tight after piercing with a fine bore needle. All materials were de-gassed and stored under anaerobic conditions ready for use within this chamber.

The feces of each donor were processed separately and, in each case, experiments were set up in duplicate. Each sample was homogenised in sterile, de-gassed PBS to form a fecal slurry. Other materials included sterile, de-gassed 119 DSMZ culture medium, a planktonic growth of Methanobrevibacter smithii in culture medium, and virus in culture medium.

The in vitro colon model vessels were filled within the confines of the anaerobic chamber with either fecal slurry and culture medium, or fecal slurry, Methanobrevibacter smithii, and culture medium, as shown in Table 1. The rubber lids were applied and the vessels were sealed. Thereafter, the seal was pierced with a 25G needle attached at all times to a gas line carrying a mixture of 90% hydrogen and 10% carbon dioxide at 1 bar above atmospheric pressure and gas was delivered to each vessel for a period of 45 seconds before the needle was withdrawn. Thus, each air-tight vessel in the in vitro colon model contained 20.2 ml of liquid under a hydrogen-rich, anoxic gas mixture. The vessels were incubated at 37° C. for 24 hours after which 1.0 ml of gas was withdrawn via a gas-tight Hamilton syringe for detection of methane to ensure all vessels contained viable growths of archaea. Using a syringe and 25G needle inserted through the rubber lid of each sealed vessel, either 1.8 ml of culture medium or 1.8 ml of virus in culture medium were added, as shown in Table 1. The vessels were incubated further for 48 hours at 37° C., after which 1.0 ml of gas was withdrawn and transferred into labelled and sealed 10 ml Hungate tubes ready for analysis.

TABLE 1
Table 1: Initial in vitro colon model liquid contents at the starts of the 24 hour and 48 hour incubation periods.

| | | M. smithii | | Initial | Virus | | Final |
|---|---|---|---|---|---|---|---|
| | Culture | in culture | Homogenised | liquid | in cuture | Culture | liquid |
| | medium | medium | faeces | volume | medium | medium | volume |
| | (ml) | (ml) | (ml) | (ml) | (ml) | (ml) | (ml) |
| Faeces Only control | 19.2 | 0 | 1 | 20.2 | 0 | 1.8 | 22 |
| Faeces + M. smithii | 18.1 | 1.1 | 1 | 20.2 | 0 | 1.8 | 22 |
| control | | | | | | | |
| Faeces + M. smithii + | 18.1 | 1.1 | 1 | 20.2 | 1.8 | 0 | 22 |
| Virus | | | | | | | |

| | |
|---|---|
| Setup at start of 24 hours incubation period | Set up at start of 48 hours incubation period |

Using a gas-tight Hamilton syringe, 200 μl of gas were taken from each labelled Hungate tube and analyzed separately via gas chromatography (GC). By subtracting the GC reading for methane generated in the Faeces Only control for each fecal sample, the contribution made by the Methanobrevibacter smithii spike could be elucidated. Having corrected for methane in the Faeces Only control, the GC readings for Faeces+Methanobrevibacter smithii could be compared with the GC readings for Faeces+Methanobrevibacter smithii+Virus, thereby determining the impact of virus on methane emissions in the in vitro colon model.

The data in Table 2 show that each faecal sample cultured in the absence of spiked Methanobrevibacter smithii generated detectable levels of methane. This indicates that each of the three fecal samples were endowed with intrinsic populations of methanogenic archaea and, by inference, the human donors of these faeces carry methanogenic archaea in their gastrointestinal tracts. The feces of Donor 1 and Donor 2 displayed the highest and lowest innate ability to generate methane, respectively. As shown in Table 2, the presence of virus reduced the average GC reading for each of the donor fecal samples. Having corrected for methane emission caused by intrinsic populations of archaea in the fecal samples, it can be seen that the presence of virus caused an average reduction in methane emission of between 12.16% and 26.13% under the conditions of the experiment.

It will be appreciated that the in vitro colon model used in this experiment is a representation of the anatomical and physiological makeup of the human colon in which ingesta transit times of up to 72 hours are considered normal. In constipation-predominant irritable bowel syndrome (C-IBS), colon transit times of over 100 hours are commonly experienced. It will be further appreciated, therefore, that the benefit imparted by virus on methane emission in vivo in the human colon may be far greater that that demonstrated in the in vitro colon model over an incubation period of only 48 hours. Furthermore, the experiment depicts a scenario in which a naïve colon is provided with virus for the first time leading to an initial infection cycle and lysis of the target archaea. In the event of repeated daily dosing with virus, as may be appropriate for a patient with C-IBS, the contribution of infection cycles already established from previous doses would give rise to a more comprehensive methane mitigation effect.

TABLE 2
Table 2: Gas chromatography readings for methane at the end of the experiment and analysis of the generated data

| | | | | | | GC reading due | Difference | Percentage |
|---|---|---|---|---|---|---|---|---|
| | | GC reading at experiment end | | | GC reading due | to M. smithii spike | in GC | reduction in |
| | | | | Faeces + | to M. smithii spike | in Faeces + | reading due | GC reading |
| | | Faeces | Faeces + | M. smithii + | in Faeces + | M. smithii + | to virus | due to virus |
| | | Only | M. smithii | Virus | M. smithii | Virus | (B − A) − | ((B − A) − (C − |
| | | A | B | C | B − A | C − A | (C − A) | A))/(B − A) |
| Donor 1 | Replicate 1 | 965.02 | 1135.82 | 1365.34 | 170.8 | 400.32 | −229.52 | −134.38% |
| | Replicate 2 | 1215.97 | 1621.78 | 1322.15 | 405.81 | 106.18 | 299.63 | 73.84% |
| | Average | 1090.495 | 1378.8 | 1343.745 | 288.305 | 253.25 | 35.055 | 12.16% |
| Donor 2 | Replicate 1 | 566.07 | 2247.04 | 1941.84 | 1680.97 | 1375.77 | 305.2 | 18.16% |
| | Replicate 2 | 427.94 | 1962.04 | 1427.05 | 1534.1 | 999.11 | 534.99 | 34.87% |
| | Average | 497.005 | 2104.54 | 1684.445 | 1607.535 | 1187.44 | 420.095 | 26.13% |
| Donor 3 | Replicate 1 | 1047.38 | 1911.93 | 1205.74 | 864.55 | 158.36 | 706.19 | 81.68% |
| | Replicate 2 | 622.72 | 2464.23 | 2758.8 | 1841.51 | 2136.08 | −294.57 | −16.00% |
| | Average | 835.05 | 2188.08 | 1982.27 | 1353.03 | 1147.22 | 205.81 | 15.21% |

### Numbered Embodiments of the Disclosure

1. A virus capable of inhibiting the formation of methane in vivo in the gastrointestinal tract of a human for use in a method of reducing the production of methane in a human subject, the method comprising administering the virus to the subject.
2. A pharmaceutical composition comprising a virus capable of reducing the formation of methane in vivo in the human gastrointestinal tract.
3. The pharmaceutical composition according to embodiment 2, in the form of a liquid, syrup, capsule, tablet, bead, granule, pellet, lozenge, pill or suppository.
4. The pharmaceutical composition according to embodiment 3, in the form of a tablet.
5. The pharmaceutical composition according to embodiment 4, wherein the tablet is coated with an enteric coating, preferably wherein the tablet is encapsulated with an enteric coating.
6. The pharmaceutical composition of any of embodiments 2-5, wherein the pharmaceutical composition is configured to location-specific release the virus to the human.
7. The pharmaceutical composition of any of embodiments 2-6 for use in a method of reducing the production of methane in a human subject, the method comprising administering the composition to the subject.
8. A method of reducing the production of methane in a human subject, comprising administering to the subject a virus capable of inhibiting the production of methane in vivo in the gastrointestinal tract.
9. The substance for use according to embodiment 1, the pharmaceutical composition for use according to embodiment 7, or the method according to embodiment 8, wherein the human has irritable bowel syndrome.
10. The substance for use, pharmaceutical composition for use or method according to embodiment 9, wherein the irritable bowel syndrome is constipation-predominant irritable bowel syndrome.
11. The virus for use, pharmaceutical composition for use or method of any preceding embodiment, wherein the administration is oral administration or rectal administration, preferably oral administration.
12. The virus for use, pharmaceutical composition, pharmaceutical composition for use or method according to any preceding embodiment, wherein the virus inhibits the metabolic pathway of methane production in the human gastrointestinal tract.
13. The virus for use, pharmaceutical composition, pharmaceutical composition for use or method according to any preceding embodiment, wherein the virus is an inhibitor of methanogens, preferably an inhibitor of methanogens in the Methanobrevibacter genus.
14. The virus for use, pharmaceutical composition, pharmaceutical composition for use or method according to any preceding embodiment, wherein the virus is an inhibitor of Methanobrevibacter smithii.
15. The virus for use, pharmaceutical composition, pharmaceutical composition for use or method according to any preceding embodiment, wherein the virus is a virus which targets metabolic processes involved in methanogenesis.
16. The virus for use, pharmaceutical composition, pharmaceutical composition for use or method according to any preceding embodiment, wherein the virus is stabilized.
17. The virus for use, pharmaceutical composition, pharmaceutical composition for use or method for use according to embodiment 16, wherein the virus is stabilized by immobilization to a substrate.
18. The virus for use, pharmaceutical composition, pharmaceutical composition for use or method according to embodiment 16 or 17, wherein the virus is immobilized via its head.
19. The pharmaceutical composition, or pharmaceutical composition for use according to any preceding embodiment, wherein the composition further comprises at least one additional virus capable of inhibiting the production of methane in vivo in the gastrointestinal tract of a human.
20. The virus for use, pharmaceutical composition for use or method according to any preceding embodiment, wherein the method further comprises administering at least one additional virus capable of inhibiting the production of methane in vivo in the gastrointestinal tract of a human.
21. The pharmaceutical composition, or pharmaceutical composition for use according to any preceding embodiment, wherein the composition further comprises at least one additional active substance alleviates the symptoms of irritable bowel syndrome.
22. The virus for use, pharmaceutical composition for use or method according to any preceding embodiment, wherein the method further comprises administering at least one additional active substance alleviates the symptoms of irritable bowel syndrome.
23. The virus for use, pharmaceutical composition, pharmaceutical composition for use or method for use according to embodiment 21 or 22 wherein the additional active substance is an antispasmodic.
24. The virus for use, pharmaceutical composition, pharmaceutical composition for use or method for use according to embodiment 23, wherein the antispasmodic comprises hyoscine or a pharmaceutically acceptable salt thereof.
25. The virus for use, pharmaceutical composition, pharmaceutical composition for use or method according to embodiment 24, wherein the viruses are stabilised.

The foregoing disclosure provides exemplary embodiments of the disclosure and is not intended to be limiting. It will be appreciated that various other modifications and variations of the disclosure are also possible.

1. A method of reducing the production of methane in a human subject, comprising administering to the subject an effective amount of a virus capable of inhibiting the production of methane in vivo in the gastrointestinal tract, wherein the virus is an inhibitor of methanogenic archaea.

2. The method of claim 1, wherein the human has irritable bowel syndrome.

3. The method of claim 2, wherein the irritable bowel syndrome is constipation-predominant irritable bowel syndrome.

4. The method of claim 1, wherein the administration is oral administration or rectal administration.

5. The method of claim 1, wherein the virus is an inhibitor of methanogens in the Methanobrevibacter genus.

6. The method of claim 5, wherein the virus is an inhibitor of Methanobrevibacter smithii.

7. The method of claim 1, wherein the virus is stabilized.

8. The method of claim 7, wherein the virus is stabilized by immobilization to a substrate.

9. The method of claim 1, further comprising administering at least one additional virus capable of inhibiting the production of methane in vivo in the gastrointestinal tract of a human.

10. The method of claim 1, further comprising administering at least one additional active substance that alleviates the symptoms of irritable bowel syndrome.

11. The method of claim 10, wherein the additional active substance is an antispasmodic.

12. The method of claim 11, wherein the antispasmodic comprises hyoscine or a pharmaceutically acceptable salt thereof.

13. A stabilized composition comprising an effective amount of a virus capable of reducing the formation of methane in vivo in the human gastrointestinal tract and a stabilization agent, wherein the virus is an inhibitor of methanogenic archaea.

14. The stabilized composition of claim 13, wherein the composition is in the form of a liquid, syrup, capsule, lozenge, tablet, or suppository.

15. The stabilized composition of claim 13, wherein the virus is an inhibitor of methanogens in the Methanobrevibacter genus.

16. The stabilized composition of claim 15, wherein the virus is an inhibitor of Methanobrevibacter smithii.

17. The stabilized composition of claim 13, wherein the stabilization agent is a sugar, a preservative, an antioxidant, a dispersant, a thermal stability agent, a buffer, a polymer, an amino acid, or combinations thereof.

18. The stabilized composition of claim 13, wherein the stabilization agent is a substrate and the virus is immobilized to the substrate.

19. The stabilized composition of claim 13 wherein the composition further comprises at least one additional virus capable of inhibiting the production of methane in vivo in the gastrointestinal tract of a human.

20. The stabilized composition of claim 13 wherein the composition further comprises at least one additional active substance that alleviates the symptoms of irritable bowel syndrome.

21. The stabilized composition of claim 20, wherein the additional active substance is an antispasmodic.

22. The stabilized composition, of claim 21, wherein the antispasmodic comprises hyoscine or a pharmaceutically acceptable salt thereof.


SEQUENCE LISTING (1 sequence)

SEQ ID NO: 1 - DNA, 38790 bp, unidentified
  source 1..38790: mol_type = genomic DNA
gtcctggtgtttctccaaggtctacttttatgctttcatttttctctaaccattttgtgttatctcccgaaccgtttaatttttgccagcacccaatcaatacttcttctactgattgttcgctagtttcaaatggttctaattttggattacaatcacatactgtgagataattgaaatctacaagtgttggtaaagcatgaatatataattctcctcttggttttacataggggaatcttgaaaattcaaaaagttttgttgtatcacatgttaatgatacaaaatcacatccttttagtttgaaagttacgttgccgtaccagtagttgaattttgattgttttgtgattacatttactatattttgttttgaatcactttcatcaccagtatattctgcagttatttctttatattctcctactttttctttgactgtttttgttgcagtggttgttgtttctgcttctccaggagttaatttaacttttttacatttaccatgaccccatttgtctcctccacagtatccgcagtaatctgcgtcacatcctccttttttcatgctgcaagttatttcgccttcgtacacttttttaggattgtcggttaatgttccagttttgccacagtttgggcagtagtttttccaggtttttgtgtatcttttatatgctgttcctgcacaacagccacatgatggcatcatgtttacggttattgtgttgggatctcctgttgtgacattttcagttacctgtttttctacttctttttcaacatatttcatgaattttattgacttatttttgtagtcaatagttatttcgctttcatcctgaattagattcggttgttggaaatatatcattgaagtattgggatcatcatctgctattccctgattagttatttcaattcctagtgtggatgctccttttatttttttaattttgagaatagggtatgattcgctgatatatttttcaacatctaaaatagtacatcctgggatttgacgtggtgtaatggttgggattttatttgaagaggttgtattttgtgtgtacatgaatggcatgtattcgtagttttcataatcttttatatagtgaatattttcttgtacaaatgttaaagaaacagtgtctattatttttagttcattagaattaaagaaaacatctttttttgttgttaaatattttgcaagaacatatatcaaatattggaagcttagtcttccacctgtaattgataagctgtttggtgcttttgtttgagttatgcagtacgatttgattattcttgctaattgtattatttcatcctgttttatgatgcagtcaatatggctgtgtgatggtaaatctgcacaatctacaattttaagtgaaatgttttcagagttattgtcttcagtgttgattattgcttctgcaagtagaagtatcatttgtcccatgcctattttaacattgtagactttgttttttgatgaatcttcatatatttcactatgtgatatttcaaacttgtttgggaagtatttttgaacatcaagaagtactaatagtttttcagcacaatcaataatgtcttgtatgtaaaaatcatattcaaataatgttggtggaatattattgtttacgtcaagattgtaaaattgtctaagtatttttgaataatctacttcatcagtattttctgaagaaattactggcattatacctaatgcatttttggtttcatcactactatatgataattcattagtattttctccaattacaacactttctacaattttattgtgtgttactccataatttgcaggttttagtaattgtatttcttttataatgttattattttttaaagaataatttgttttaaatactaatcctgattgtttttcaatagcttttaataagttgtattttgttattgagccttgaacattgattaatcttttactataatcaatactgtcaatatctgtttttgtaacattgaaaaatcctttcagtaatttgtttaggaataatttactgataatgattgtatttccatttacatgtgaagcataattgggatctcggatatagaatggtttacaattgtttaattctacaattatttcttctgcatctacatcaatagatttgtctatataatcaaaggtgcattcactattaataacaaacaaacaattatcaacaaagattttgtttccttgtttgaataattgcttgtctcttttaacatttgtaagtttatgtgatagttttaatgtttcatatccttgatattcatcagtaatatgtatttctgataattcagtatctaaaaattgaagaatattttcttgattatctaaaacaacaaccatactttttatcctcctttttcatagtaaattacatcagtcactttacaattgatactgttttcaaaattaaaatgatgatccaatatgaaaaaactagaatctacactaatacaattaggatctatcttgaaccattcactatatttttcatagaatacatttctattttcacaatcaattttaatctttgttgtttcaggtaagtttgttataaactctccttcaagtttcattagttgtccagattctgattcaattatttgtatgcttgttgattcatcttcacttaatttatacaaaataatgtctggttttacttttccaattgtggttattgttcctgcgaaactttttcttaatgggttaatatttctacttaaaccactagggattattaagttcacttcacaatcataacctccaaccattgcttctgcatcaattgaatcttctatatagtaatcaaaacattcttcaggtgcgaaaaagaaactaatactttttagaattggtgaatctaatgtgtctctttcaggatacaggtagtcacttacatatttcatgatttctgttgattcttcaaaatcatctccgtaaactctgaattttaatttgatttttttatcatcaacattgactcttatagggtatttcccatctgctccttcaattttatatttttctgtatcaaaattagctccacgaggtatttctgtttcaggactcatacttataaggaaatatttgcaactaacaccattaatatagaaatcgcagtcttctccactattttttgaataatatactgtgatttgcacatttttcatttcgacttggacttgagtgacattgtcaaatgcatcatcaatttgtaagaaaaaccttaaatcatttaatattgtgttgatttctggaaatggaatgccgaaattatggaattttccaccgaatttaaagttagtgtcatttctagtgatgtttatgctgtctaaatagtaatctaattcatcatcatctaatgttccaacaccacaaattagattaattgattcttcaacacttatgtctccagttatttctattccatgaatcagtacattattgttttctaatccttgccagttgaaaccttcaaagaaatgttttgtagataaagctaactctggaggttcaagtaataaatttgcagtgctgcctacatcacctaacaaatatctttttggagataaagccaatactggatattcataacctgcatatctttctttaagtattaaagataaattaccaaattctacagttccatagtcaaaatctatgaaattcccataaaaaattagatagattggtttacttgcatcatatgtaaactcacaggataactcttcccatatgtccttaacactgatttgattggtgtatgaatttgttgttccatttacaacacaaactcttaagtttttcaaacctttttcaattttagaagcatttaacagtcttcctaatgctgagaaaacataagtttcaccatgttcaaggtttgataatgtgaattctgataaattcaatattgaataataggatttatatgaatcaggaatgtttactttcactttatctgtgaaaacacctaataataatggactggtcatttcaatattatgctctccactacttttagagtataaaggaatatttgtatcaaaaacaatgtttccactatcatctttttgccattgtgattttgaaatttgtaaaatattatttgataatcctggaacaggttcttttacagttaatcctccaaaattcagatttagtgttaaatctaatggagcatcagtttttaaagttatgtttaagtatgttaaatctgtaactgtatctgtactaatttcaatttctttgttatttgcatagaaatcccatacttccaattggggtttttctactacaaatgttgtttttgttgaatggccactattgttttcatcaagtgtgattgtaaacaatccttcagatgttggagttacatagaatattaattctgctttaccatttgttgttgcttgtctccatatgcctgtggtccagtcataatatggtgaatcatatgtgatttcacagtctccatttgtgattatctttatgtttgccgatatttcttctgaaggttttgttccactggtttcaaccattgtgcattttacagctactttttcaccgtaactaacaatatttggttcaatgctaatgcttggaatataatttccagttgtatacactacaattactcttaagtatttcatttcaatgtaacatgggtttgtagatgtattttgtggtaaatcaaatttaacatcaaaatttggagtgttaatgtcttttccttttagattcaaattattaaattgaacttcatattctccaaaattatttctaggtggggcattacctgtttttgacaggtttaaactacttaatatgatttctggatttgaaaaacttccatgtgcagtttgacttatctgtgcagagtatgctaattttctatatgaatattgtactattatactcgttactttagctgaatctggaattaatccatcatatttaaaatcatatccacgtatggttgatggatgttttcttgaaccatttcttccagcaatcgcatttttatattttgctccaggatctgcataaccccaatgtcccacagtatttaaatcatttgtaatattttcaatatgatcacatgaacgataaggtatgttgtttccttctatattttgaactttactaggatatcttaaaatactaggcaatttaataatccctccatcattttttttaatttttaataaaaccatcatactctgaattaaacaataaacagtttaattgaaagctaatgtaatctcctttacttaccccatcaggaaaagcaaattcaacactcacatatttagcaccagcaggtatttttgatgttttaatagcacatcttcctttattacccataccttcattagcaatatccataatttcctgattttcaattaatttaccatctttatcgaaaaatacaaacccaacaagtatgctttcaatagtgttttttgcatcataattagatgaaaacacaatactggaagcatcatcaggtatgtgtactccttgattgttctgatggattaaaagaaaaaaaggagaagttaaatcaacattctccgagagatatgtttctttaactttcaaagtataaccatttctaaaaggaacaacttcaaaaaaatcactatatgggttgaaattaaaatcaaaatgttcacaaacaaacaaactagataaactagattccatacaatattgaatatcttcactaaatctgttagttaatgaaacatctttacttaatttaaatgaatatacattgtaagattcccattcatcaggttttacattatttaatgaaaccatacatttatgtgattcaaaatcgtttttatcttccagatatccatgtttgttacttatgatattaccgaagatttcacattcttcaggattaatttttatagtttccattatcttttaacctctcattcttttaatacgattgtctcttttaattaaagcttcagcaatgcggtcaattaatttggaatcagttatcacttcttttaaagttgtgattattgatttttcatcaatatcttctccttcaacagtaacattaagattatgatctattgttaaatgcacatcatcagatgtataagacactaaattatcagcagtagtattattcctattatgttcaatattctggaagtatgcattttgatttgttttcattaaatcaataatattatatattccatctgtaatgattccgaatacatctttagaacttcctccaggacttgctgaaccaaaactaactccagaacctccgcggaatattcctccacgtttttggaattgtgtcatatcatatatttgaccattaaccatagctgctacatggcctattccattccatgatccatgaatcatgtgtcctgataatcccaacatagatgctatttcaattatcatttctgcaccatcaaaacagttgcatcttacttgatcccaaacttgctgattactatatcttgaattataataaaattcataagttccagggttttggaatcctcttgcagttaagatttttcttagaagattttcaaagtttccaggatttaattgcggttgttttccatcacggaaatcttccacatgataatctgaaggtatttgaattccaaggaaccatgggtctgctatcctccatttgtatgcagtattcattattttattcacattagtatcaggaattcctgcaaaacaagtatttggattaacacaaccattaactcttgccaaatcattaattacgcttgttggtaaattataatctaaaagatttaaccatgatttgcttttattgttttgaggatgttttactttagaattagtgtcaattactctgccttttccttgtcctaatcctccagcagcataacttgctattgtgccttttgtggattttccaaagttaatgcttccaccaggtaatgttctgcctccaattaatcctcttgttgaattagttgctgggcctgcaggcagtccactactgaaatgtgcattggctaattgattatagaaacttgcaatacttctatgaaggctgctgaatttattgtatgattgtgtttgtatgcttcctgcagctgaaacaatattgtctttcattactccccatgcattagtcattttatttgttacatctattgtggagttacgtacttgatttaagttctgggttgttgttgatttaatattgttccatgcttgagtatttgaattactcatgcttgttaatgcagtggttactccattacgtgttgtttggaagcttgttactactcctgcaactctcatttgaatagctcctgcatttaatgctattcctgcagttaatgttgaaaagcttgttccgatcatattgttgcttgcagtgatgctttcactagctaatgatacttcttcaacaagaccattatattggtctccagtttcttctgtacctgaattattattgcttaaatcaacatttgcactatctgtttcaacttcagtgtttgttgttactggagttgtgatagttgttgaaatattatttaatgcattttcaacatcagcagtattgaatccatttacaatgttttttccaacatttttacctgcttcatatgctgattttcctttgtctttaactctttgtagcatgttaaagaattctagtactactttttcttggattattcctggtgaatgtattcccattgcggaaagcattccgtctacaatttttttaccgatttgctttgcattttcaactaattgtgaacctgcagatagtattcttgagcctatgtttaggaattcctggtatactttttgaggtaattgctttatccaggacattactccagatactacttgtgatgcttttgttttagcattacttacccattgtgctcctgcagttattactcttacagcaactgctaaaagatacatttttattttatctggcaattgtcggataaattgaactactcctgtaaggaagtttctaccagtattaactgcattattccacattgactgagcccaactaacaacacgattaatcacatttgataatattgtgctgattgttgattgaatagctgcccaagcccaagcaattgcttgttgaagacttatttgaccagtaattaactggctaaaaattgttgccagttgcatgaataaattgaataatggagttacaatatcaagtacaagttgccatgctggaccaaatacacttaataagaatgatgcaattccattgaaaacatcccatattatggtgagtatgtctattgcaagttgaactctaactttgaaactttcaaatactgctcctattgcatcgataactgctcttgttccatcaactttaccttttgcactttctgggaatatttcatcccatattcctttcaaccaatctacaactggtttgagagattcattcaaatcagaccatgcatcttgaattgctttgatagttgcttttacatcaggatgatttatgaatgcatcccataatctaccaatattattctttatggcttcgagcatggttcctacatctttccaccaaccaaaagctttacctacttcatatattgctactgctattgcaactaatgccacaacaacccatgttaatggatttgataaaattgctcctgcaactgctaaaaaactagccgccaaacccatatttgctatttcaacacctaatgctccagcaatataagttacaaaactagtcgcactagcagctattgcgggaattactgaaccaagaattacaatacccaacccaactaaagctgttgaaagtacaccaatagttactgctgctttagcccaatcgggcatgcttgaccatgctgattgtaatgcttcaaccatccaagtaatcatttcaacaactgttataattatgggagttattgggacaaggatagattgtattaatttgcttccagttactgttaatgctgcccaagcatcatctaatgttacaatttcttgtgcggactgttcaaaacccatatcgctaagtgttttgttcattgcttggagtaatccagttttattatttatatctccatcccatccatttttcatgagcatgtcttgagatatgcctatttcctgcagtcttctgaattggccatccattgcatctgatactgcaagtattgcatcttcttgtgttcttccttcttttacgaaagctgaactcattactgcagttgttttagtaagttcatccattgaagctttaggaagttttaatttcactcccatttctaatgcggctgcacctactgcattcatatctacttttctgaagctatcttgcattttgtcaacagccccatggaattgttgtagttctccttcagtcattcctaatctttgaccgaatctttcaaaacttgcagctgcatttattgattctcttgctccttgaaccatactgtttacaaggtcaaaaccaatcattcctacagtcatacttgctgctgttctaagaaatccaagacctccacctgcagttttagctccgttttcaagtcctccgaaaccagttcctgttttttgtgaacttgttcttaaaccttctaaagctaattgtgcttctcttgcatctcttgtaacattatctaaaccagtagtatggaagtttagcaatgaatttacttgttgaagtataccaattaaaactaataatattgcttttagtagttctgctgatgctgttgttctactgaatcccgaaccatcaattgaatctaaggctgcatctgttgaatgtgcacttgttctagctcgatctaatgccatgtctaattgtgctgcacttgatgaagttgctgttaatgtgtatccagttatactatttattccactgcttaaactaagtgcactggatgaagcttgttttaaacttgaagataataatctggtactattatttgaactattcatcgaggaagataatcttgtagcactagatgatgctgtgagtaaattacctgaattaattgtgttgattgaagtgttaagtctatatgcactattcatcgatgtatccattgaagtggttaatagttttgcagaattagataattgattaattgtattgggattgattgaatttaatgctgtttttgtagaattagtgcgatttgtaacttcagttaattctttatctaactgattagcagagtttttggctttatccatacttgaggaatcaaattgtcgcattgcattttgagcttgttgtgctgctttttgaacttgctccatttgtgcttgtatctcttttaattgagctttaacacggttttccagttcaattattaactttacaatattattgctcattattctattcctctgctaatttcttgtttttttcttctgacacggtcttttagagaatgtgaagcatgatttgtacctgaagattgtttatcctgtttatcacgaactaagggtactgcatgattcaagaataatttctgcctaatagttaaatctgcttggttttctgctaacatataaccgatgtcttgaagagttactacttcaatagctatgttttcattcttcttcacgaaattctgctatattattatccattgatttaaggttatctaaaccactgaaaattaatgcttcatttacaattaagtctaatgttcctgcttttaattgttcaaggtcttttttggtgaatttatcaggattgtctgcattgtctaatacttttaatgcaaggttcatttttgcatcatatttctttttttccattgctgcgagactggttttgaattttgtggtgtcttctgcaaggctggtttgttttgctttgatggtggaaatcatgtcgaagtttcctacatcacggtatgctgatgtgaattggttgtattcaaggtcgcttatttctcttattgctacttcattatcgtacattggtagttcgattatttttgtgtttttgattccgccgatgatcatttcttttgtaagcataaatatcacatttttatataataatttttttttgaattttaaataaagtaattgaatgagaataatcatattctcatttcatttaatctgtgtaaacagtgattgtttttgttttacttgcaccatcaattaaaatatgtgctggttctactttagttgcagttggttcaccagtcaactctatatcatacactccatcttcagcagtaagtgttacttcaccattacttgaagattcagcatcaacagttgtggatcctttcattaatgatactgtgtaatcagttgttaatttagcaccttccttatcaacaagttttattgttgcatcataagttgggttgggatcactgtctgcttttcttgcaaataatgctccagctaaatttttataatggtccattgttgattcaactgtttttgataaacaaacaatacttaattcatatttaactgggtctgcattcattgttgcagatggttggttgataatacacattggacaatataattcgaattttttatcaccatttctaatgtagaacattaattcaaagtattctcctgcaaggtatcttcttggtccttcctcacttccccagtacatttccaagtatttttcatcagtatcatctactgttaagtttaaaccaatttctcttttagctgctcttggtattttacacataaaccttgagcccattcctcttgcatcatcagtattaacattgtttttgatttcaagacttattttattggttttacatctcatttcagtccattcatcttcaccaaatcttctcattttaacactgtctatatggtaaaatgataatggtaattttccaaagttaaactctgataatttttttaatggttgtttactattgatttttgatttgatttctgctgatgcagttaagaattctgattctacttcaatacttaaactgtccatcaccatacctaaaatttccatctcgtaggttgcacatcctgccatcacagtataagaaggtaagatactactatttgtaccgtagataatatctccttctcttcttcccaatactgcttcaagaaggtattctaatgtttccatttgtaagttgcaactgaaactgttttcaggtacatagtaagcagctacaccagattgataatccctacttactactgaatcatattttgtaaattgctctggtggagatacatccatctctgatatttcaatatcaatagattctccactaaggtctttttttgcaaattctccttcttttactaatcttaaatattttagttcttctgcggctgttaccatattctttagcctccatcacattttgaattcttaattttaaatcttatttggaatgttacacctgcactatacacatttttctttttacccagaccatatggaacccatccgaaatccttggttttaatattgaaaaattctaagtctccaagtaatcttgaacttattattctacgttctgcctgtgaaactatacttgtagcttcatgaatacctatcttaggatttttattgttaagcacctgtgaggataatattatttctccttcaaaggtttcacttaaaccagtatcagtaatatttttaaatggttcatctacccataattccagataaggtaattctggtgttcccagacttacacccataagagataaatgtttaatagtgccatcttcaatcatttcagataagaccagttggaaaccttccattagtttatcataagcttctatgaaatctttcataatcccacctcttttaatgtttcagacacatatttgttaatttttgattcagtagttttaactgcatgaggataataatggtatcccctgaatgctgctacttttttaggtttagggacaaaaacatctttaccagatttatccacccaatgcagggcttttttatttttagctgagaaacttccacgaccatcatgaacatatttttcataacctgaaggttttcctcttgcttttataacatacatttcatctgtttcccttcttgcagttattgcagttaaaagattgtaacgattgtattttatcctactttgaaggtaagttttagcatctttacttgctccataacttacctttttacctaatcttggaggtacccgtaatattttttcttgcagtctttcccatcccgcataatcaaaatcaaaatgtatcataaaaaatcatcaactcttataaaaaattaaaaaaaatgtttagattgagaaaacatgaatcttagattttttaatgtaaggcttcattcttttttcaatgtcttctgtgaaaatattgtttactgcctcatcaaaatcaaaattttcatgatcagtaattcccaagtcctgtcttacagcatggcttctaataatgttactggttaattcaataatgatttgtattacatcatcaggaattttatctggtatatcatatttctttttaacccatgattccattgcagtgtaataatcattaatataattatctaattcagtagtttctaaattaaataattcttcagtatttttattagctccactgtactgtttgattttttttaaggtgttttcatccaacataaattattcctatgttttagtcattgtaatggttaatgtagttgaatcatcacctacagtataattacctgtgctttcaaagttttcataaccagtacatgttgcaccatatgaataaactccataaggaacattacttattgtacagcctccagcactaccagtaccgttacaagtgtaagttttaccatcagtaatatttgttaaagttacaatagctccttcaacaggatttgttccatcatcaacacttacaccaatatttttagtactagttgttgtgctggagtttatgcttcctttatgtatgcaagagcagatgcatcagaccatgcaaactgaatatcagcatacattgtagaagcaatataatacttgttagattttaaatcaaattcagattcaacaataatattatcaggatccgccaaccattgaatgttttctttatgagttaaaattactggttttttagtgaaaccattacgaagagtactgaaagctggaattggtactaatggtacttcctcaataatgatgtccccatctttagtaataactacatcattaacagcatacttgtcatgattatctgctacatatctccatactgctcttttaaaactgtatggtacaaatgcagctaccccaccatcatttttatatttatcagggaataaatccaacattctacggaattcttttaacggattggaatctgttgcagttaaatctattgtttcttgatctatatcagaatcatcttctaattttttcaagataccatcatttactttatatgaagttgcaacattagattcagtacttgcagtattcccatagattaatgttctttctaatgctctaccatttgcactaccaaattgaccagttaaagtattcatgaaattcttttgctcaatactgtcatataatacagttctgtgaacaccagttaaagcacgatattcttctgcatcaaaagccctattagtgaatgttgggtcctgttcattgcttaaagtttgtggtgtaccactgattcttccagcttccaattcaatatcaaaactcatcatgtccaattctcttttatgattgtgagttggaacaactttagtcttgtttaaaaatgctgtttcttcttgaacagcttgcatgtatttatctgctttttccgcttgaagaactcctttaccgagtttcccacttccttctccaatatctacaaatttaaggataaaatcttcattatgcatgattttatcagctaacgcttgttctacttgtttaattgtcattttatatcaaaccttctcataaaaaatttaattattaagtataatatcttctttttttctaccaggtcattccatttgaattcctacccattctctcaaggaatgatttttcagaagcattgccttttgcaaggtcaggatcaatacttttactaacagtagcatctggattaacagcaccatcagcagtaatagtagtatccactttaacttctcctccatcatcatcttttggaggttctgattttttaacagctccaggaacaggatcctcttttggaggttctggtggtttttccccaccttcaagtttagttatccttgcatccatagtatctaatcttttatcgattttacctagtgcttctaaaattttattttcatcttccacaggttcattattttgtgctggaggttcagttggttcttgaggttcagatttagcaactaaaccattaattaatttttcaagaataccttctgaaacctgcacattattttttccattgtcttcttttgtcataacttccacctcttctggtgtgatatattttttcacaaattcttcatcatcttcatatacttcaaaaaccgccataggatgtgatggttgatcaacaatactgattgttgatggtttccaatctttaatgtctttaaatttcaaagccatatttaacctcctaatcctctactcatttgagcaacacttttatatggtgcagccagtatgctgaaaccattgtattccccatctcggattgcttgttgtatttcctcatcagttacatcaacagaaataaaccaggatcctttggggtaagtgtttcccctgaaagtggttggtgattcaagaatatatgattctaaaattcttcctacaggttggagtgtgtgctgtacatcaactcctaatcccaggcgattgtatattaatgatgcttgtcttattgtttcttcatccagtatgtctcctgttgcatcagggattcctggtatgcagactgcacctttcacaatcatagttttttttgactccccatttttttatattggaaaaaaattagttttgttggagttttttttagtaaaaaagaaaggaggattttaaaaaaagataaatgatgttaagttactaaaataaaaatatttttttatcccccttcaatatataggagtggaaaaatagaaaaaaacgaaaagttaaataaatttattcaacaattttgaccataggtttaaattgaggatatttatcaagaaaatctaaagcatattgtatatgatattccctagcttctttgtgtttcccttcatattttaatctatgagttacagcccgttcattctgatccaaagattcaaactccggatgttcaatacactgaattaatcttaatcgattaatattaggaataacttcacgtacaatactcattaatctatgaggtaaacctgaaggtgttgtaatgaaatctcttaattgatcctcagaccaatttttaacaaattcataattaggaatcaatttttcaaccatgataatgaaccccagtatttgttagtatattatatataaaacttcctatataaaaattacgatatcatttcccaatgagcaataaactctgatttttcacgtattaatcttaaatcaacatttttatcaaacaaaatttcaaactcctgaggagtttcagttattccttcaatatacagaattttaattccttttggaatgacaatataattaatatactccccatattcttcaggtttaacattttcggaaatagaagttgataaaaatgaatctgaatgataattatctccttctacaaatttagtcattggacttttttgtcttctaactacaacaagactttcttctaatattttacctttcttaaatactgattttgaagatttaattgctttattaaaagagatataatattttgaattagctaaatcctgctcccataatttagaatattttcttttaatacgccacctcttaactgggttgagctccccttttatttctctgaaataattattcaaactataatatccttcatcagaatatgcttttatgaatttatactcttcagaagtcaaatcttcttttttaattccatatttatattcaaatgccaatttatctttagatgattcaccaacaccataaactgttttattaccaattttcactgctttatttttagctttactaaatttagtgatagattcatttaatttaaatttactgaaatctctttttgaattttgttttggttttgaagaaatattaatcattactgtttttcttttaacattttcagtgattttacttcctgtatttttaatcttgttacctatattcgatatggttctaattatcttattcttaatcttagaagttatcttcaaagaattttgattactgctacttttattcttaccatgtaagtaagaattagaaggaatattaaaagaacttttacttttaagattacttggagtactattagtatagcttaaccaacatctgcaattagcaacattctcagcacctccagctaaatccccaggatacatcatctcagtacgataactaccagtaataataaaataatcatcaataggaacactctcaataaagttagcacgatgccaaattctatgttgtctagcacgaccattcatccaaaccttatacttataccccttatccaaagcatgtaaaaaactcatcctagactgattagtatgcaaagcatcacgaacaatattcttagcattcttacgatacaactccccatacttctttttaatctcactatcagacaacttactttcagcttcaatcttagcttttaacttataagcatcactatcaaaacgtgaagataaaatcttagaataatccccaacagtttgatcaacactaccttgaataatcttagaacgctttttcattaaagtattagcttgaccttgcaaagcagtatcaattatgttctctgcaatctttttactattcccaattttatttccatcaataatattatttaataatttatctttataagaatcaatacttttatcagctttattcgtatgtttaaacaaatgattaaaacttatatctcttttaaccacataaagctgatgcaatctttgagttaaaagcagatactgaagtttctttttagaaatcataataaaccattcttattaaaatcagaattcaaagcatcaatcatactatccaatttaccttcagcatccattggagcatcaccatacaaaaccttatccaaagactgattattcataaacctacaattataatactcatcatcttcactcattttcaaatcaaaagattcaccaaaacgattaacaaactcccctaaagtcactgcaccattttgcagtaatttaattccacgatccaatactttatcttcctcatcaaaattaacaggcaaatactcaagtttatgtgtaaatattccaaattccttttgaataatagtcttattaatcaaattagccatcctcttttgcaatgtagctactgtagatttactataatttttaagcaaagtttcagtacgattactagcaatacctgtagactcagaatcacctaaacgttcacgaggtacacgatgaatacgacgaatacgatcagccacactacctgacagttctaaaaaactgccttcttttttctcatcagcaattttagtaacattaacactaacattattttcttcactgggaatagtgaaaactaaagcagttccaggctcattggacacttcacgaaactcatgttccaaatcctcttcaaaatcatcaaaagtataatcttctttttcctcaacattacctgtaacagtaataatataattcggaataccatgtgctttaaaatgcccttgctgatactcaataatagcattatcagttaaaatagcatccaactcggacaaatatttcggtctaccataaacaagactttcattacttttaagattaaaccagattaaatcatttgcacgattttcatctgtaatattatcatcccacaaaccagtttcacgattcaattgcttaatattgtcaggatcatataatttgaaataattttctttatgccctatcttttgtacaacacgttttttatcacgacacattctcaggtataaactgcttacatgattaacacttttcaactcccctttttccctaagtagttctaatcctgcaaaaccaaaagcttctaaatcttctaggaatgattctatttcctcatcaaaattaaaatcatttaaaaaatcatctaatccttcaggtatttcttctccttctttagagattatttttttacctgtgaaaattgcatcttcacatttaacagtaatacaaatatcatgcaaaccactaatatcacgcaacttatctaattggaacgggtcatacgctgggtcaattacttctgtaccatatgttaattcatctttactcatttcttgagatttaatctcatattcatttaaaacacttttaatcactgaatctcttaaaaagttacttttaactattttaaccattattaatttctccttctacgatttctttttcttaaaactgtaggttttgtttgaggatataaaccttcatgtaacaatgatacactatctacacggtcatcatgtgttgtttcatcatcagctattattttctctgatgggaatttcacagcttgtttcatgaaatctttcaaccatttaccacgaacaaataatatccgtccattattcatcccccgtatagttctacttgctcttattaattttgatttaggaacacgaataaaattaggatgatagtcttcgaattcgttttcccagtatcttttaactattttacctgcagctgcaggctgatactcaatccaattatcatactctgaatgttcatccataatccttgtcatgtattgttctaacttacctggctttttttgtgtgcttttctgattatgcactacacctacttttccttgtaatactgttgaaaaactacatgctgtataatcagaagtacttttttctgttgctgcaatatcccatgtaattacttcttgaagaatatccttttcagttaataattcattaaactcttgtttgctaattgttgctgcttcaattgtatcataatcaaatacatctccagctcttatttcataatcccaattaccaatctgatactgataatctgcttttgataattcacgtaaattagctaagtatttttctttgtcaatatatggattatgccagaaattcatttcaaaaaaagggtattgtcctttaacaaatttttcatttaagtatgttgatccatctgcatcagcaggattactaatatagtaaattgctaatgggaaagtcattaacttatcagttcctctgagactacgattcaggaattgcaggtttactttttcaaattctgaagcttcatcaacaatgattttatgataagcacgacttttgaatttctctttgtctttttccagaagcatatatgaataataaatccgtgcatcattttcatggttaatgaaacatcttttactttgattgtgttctatgtaatcgaatggttctgtccaatggtccaggtaatctactattcctccagtcgctattacattatcataagtagaccgtagaattaagcagcggtagtatggtacttcataatgttgtaaggctaatactgctccaagcatacttttaccagaatatgcggaacctccaattagttttcttgtgtggcggtctgcaattgcatataattgtcggtcgtatggtgttactggaatgtatgggttttcaagtattgttcttctaatcaactttttttgttgttgatctaagtgtattttcttgtaatctactctcataattcatcagtgaatttttctaaatcatcgttaatggttagtaattcttctaattctttgtcgttcatatggattacattatcattttctttggtggatgtttcaatacttccttgtatttgagtattattatctttaatatctgttggttgaccatgagctagtctgaaatttctatagattatttccgcagatttatttagatttagaaaactatttgctcgtgtagttggagcattctcattactattttcttttagttctttttgtatttcttttagtaattcaaaatcatcattgaagaattcttgaaacttagcatttccttgtttgaataattcatagttccactctctttctttttgatccatatgattatcatataatctgcatctttccacccaattccatcttgaagagagtgtttttagttgacttagtgaaggtatgtttttttcttcagtatgcttcgatttcttcaattttattatctgtgggaatgttcttttggaacctaaatctctgtattctttgaatagtgaatagcttttacttggttctcctttttgtctttcccatgcttcagtcataatccccccatccttatattttttttgaacattattgtaatgtttattcttttttgttgaacacgaaattttaaaatacgggattttttattttcatagtgaacaatataatattgtttagttttgaattttgaacaaaaaaaagttttattagttttttataatagttttaggaaattcacaataacaaactgaaatccatcaatcaaaaatacaatcaaagcaccaacaattgctatgaatactccactttttattgtgaataatcgattatcatcattagcttgttcttcctgaatagccaactgttttgtttgaatttcaatacttttatccattttctttatcagtttttctaattgcttattttgaaactgatcacttgcttccagtttacttatacgagattcctgtttacaatatttctcatgtaaatcttgaacttgattatgggtcattattgtttcctccattgttagtggaggtgtaagttcttgaaatccatccaactatcccccctaatgctactgctgctagttcgttgtttccaatataggtgcttagaattcctataattattattccaattattgctaatgttgtgttgttgaagttagtcattattattttcactcctaaaaaaaatttgtttttaaaaaaaacattggggaagggatttgaacccctgcgatactagtgtatcattagattagcagtctaacgccctaccaggctaggctaccccaacattgttggtggagggaattgcaccctcaacttttgagtggttactaagttacaataaaaaataagctattaccaacatcatgaacatgtattaaaggagataaatatttatattatatttatgggaagaatatttttttttaagatatgtgatattaattatttaatctttggaggatacctatatattttaatgaagaaaaaaatccaatcattctattattcattaaaaatttgctaccattcccataataatttttaagaataataataaatgaaaagcgaataataaacggataagaatattatttttaaattgtcaaataatgataatgcgaagctaattgtacaataacatatgataaaatattttttataagtgttttattacaagaccacattttttacaaacatactcatcatgccactcatcatagtaaacctttttgcaatcttttttcagtttttcacaaacaggacactctatttctgcatgtttcattttttaagggaaatacacacccgcagtttgtgcaattatattctgcacgagattcatccaaaacagttaaaggggattcaggaaacttaatataacaagatgtcttatgatttctataatgtttttcttgttttttcctttgtttccacttccagttacaaacaggacactctatttctgcatgtttcatgtttttaaaaaatgtattagcatccaaataatattatcctcccatatataattataataatgtcttttatttttttttcataaaaaaagataaaattataaaatattttatccccttcaatatataggagtggaaaaatagaaaaaaagattaaaaataaaaaaaatttaatcacttaataaataactatcaagaaaatgctcaccacgactgatatgtcccttacttacaatccatttaatcatgggatccatcaaacctttttcatattctttaataaaataattcaatgtccaaaaagtaaccttttcattaaaaattatcctttcaaaaagttctttagatgtctttttaaatttttttctatgaaaaataatactgtcaatttcatcaatagttaaatacaatgcaatatcttcaccagaaatataaatacattccttatcaacatcataccaataatacttctcattaggatttcttaataatttaagatacttgataaaattcatatttttactcctattctttttaattctcttttcacagttctttcctccaaaccaaaatcactcaaagcatgctgtgaaagaaatgttgtaccaggctcccttaactcattactaattaactccccatcattaattaatttcctacgtttttgttgatactttgctttctgctcacgtaccgcccaggtcctgcaagcttccctgcaataacctgttttattctcaaacttaataaatactctgccacagtatttgcatttgctgatgtagaatcttgtttgtggatcatttaatgttttcttcaataacatcctatcatctttctaaatttttagatagtaagtttttcttcacttcaaaaacaaacatgtgaaaaggtaaaagtttaccctgatatttcacatattcattaattacttgtgatttttttaatgcttcagttatagtttcttgttttaattcttcatttatgcattcaaaattggagactgttctacttaaactaatgtatactctttcatatagattaaggttattgtttttcacattctccaccacaacaagaaactgaattttcagaagtgttgaatttaccatcttcaatcatttcaatcaactcattaagaatacgagtaacatgcatcaaagtatgaagattaatactatgaattataacatcatcatactccatgtctctcaaactacctgtgcattctttatgaatagtaaaagattgttcttcaaattccctgcttcttgttttaataaaatatagtaataatttctcatcaatcatacatatttatcctccatgtattttttaacttctttcataaaaactcctgattacaatataacctatcttaatcatcaaaaaaatcagtaagacataacactaaacaacaactaataattacacttacactaaaaactacccaaccatccatcatactatattctccccccattatctccaacttgttattaactcatactcctcatgagttaacttctgttttaacctttcatcaaaacttcttacatctccaggattattctcatcccaacaagactcaatcttggcagttaattcattaatttcttccttattcattttcaatatcctcctatttaattttgaaaggttgtaatggtagtgttggtctttcaaatataatctgaaacaaaacagtatccccacatatatctggataaatactttttaactcatgtttcaacaaatcaacatgcctataaccttcaaaccaagcatgattatgtttcaaatctttaaatttaacctgttcaactctccgtactacccgaagcagacaaaaacccgtcccctcaaaccaacattctacaaggtcaccttttttcaatcctttatcagattttcttattgtagacatttttttcctacttcttaaaggttcaaagaaatacggtttaaacttcaatgttttcatttttatcaacttccttttttacatatctttgcttcttttttatcatcatccaacactatattatgttcttttaggtatgattttccaaataattcttcagcatgtttacataacagataagaatcaattactccttcagcagatattattccttctttttctaatttttcaagtatatcattacaactcatttttggatctcctttttattccggactggttgtctttgacttggttcaggaacaccacacctcatacgtaccttttcagcaaacttaacaaactcatctgcaaataaacttattctaaaccacatcaactgccaccttaatataatatgttcctcatcttctttcttcatgattccaccttttttaacctagtttggaagttatttcccccataacgcctaacatgatcttcacaaatgattcttttcacaaaggaatctttttgatgtcgagttaatccataccttttctgcaactcatcaacagagatatttaattcatgtttataatctctcctgaatttcaaatatgtttcttcacctttagaaacatcataatccgtttcaacaatattaaaaactggtttattcattttttatctctccttaaaaaaatgtaaacataaatgtttacaatttaaaaaatatttatagtgaattacgtacgtatacgtacgtacgtatgtgcacgtacgtggtttcctacaggttcttcggaaaaatgtaaacataaatgtttacaaatcattttatcaccaaaccataattccgaacatcataacccaaatcatacacatgatccaataatgacttatacggaacttcttgctgcttactaatattcttcagctgatctttaccaataaaaccagcattatcaacaatcctattaactgtacccatcaccccatcaaagacatttacacttctttcatgttctaaacgctcagcacgaatcttacataatcttgattcacgagcattaatctcattttgcaactcagctatctcttttctcaaactattttcctcatcttcagataaatcaatagctaatcttaactgctcttctaaaaactcacttctggttgtccccagtttgtttttagcttccctccatacttcagaatcaaccatgaatgaaacctgtattttttcactcatacatcatcacctaactctttcatgacagtttctaattgcatttcctttgcaattaaatctaattttaaagacctgatttcctcttttaactggtccttttcaattaacctttgattttgtttactcacatacatatcaatgcaatgttccacgcagaaacgtacagttaaaccatacttttccattaacttttttgcccttgtagtaatcctaccagataccacttcagggaaattatcctgagtattcatactcttacacctaacattttttctgcttttttaatatgtttgcatttataatgttctactttttgatgtcggtagaaataatcttcacaactacaccaccatccatctatactgtcccagttcacagtattggatccagtatttcctgatgccttgaattgtatgaatattatttctaccttaggttccataattaaacagcaacctctttttcttgatttttaaaccagttatatgcttctttgttttcagctttggtcagttcaccatctttgaataacttcattctatgagtattaatcatagatggagttatttccagtgaactgtcctttttatgaatatactcgcggattttctgcacagcaccagtagcctcaactttctcagttcttgcagaactacccttagcagttttaacattaattttatcagaatccacaacatctttttcaacaattagaaacatgtttgctaataaatatctttttaagtaagtaatgtaactgccttcactttgcattacattcatacctttatttaaagcaactatttcaggcataggaacactggtgatgacagattccccaggttcattccaatttctgattttcaattgagccaattcatttgtaaatgaaaactcaatgaataactcctgttcataacattcacaaaagattgcaggcaaaatatcttctaattcatagtacttaaatttcccaaatttattgtacccacttttaggtaattttttatgcaggagattcttttgaattcctgcaattttctgatatatagtcataaaaaaataatccccctttttacatattcatcgactttttcatcaaatccctaatttcttttaactgttcattttcacttatttttctaaacatggtgttcagaatatattcatcgtaatctatatttttttcagcacataactcttccatcagtgctttaactattctaacttgatttttctgtacaatatgtttaaagatcatatcaaaaaatgaattcaattctgcaggatttttagcatctacattatggtctaaaaagtggattgcattagttaatttctcctttaactcttttttgtttatgatttcttcagtaaatagttctttctccattaaaattcctcctcaataatctccaatactttgttttcatcaatattttccaaagatgcttgagagtgtggaatgaaactttttgacacccagtagtcatgatactggatttcaccaccacacagttcctgtaaagtattcataatatcacgtgtgttctcataagtactgtcatgaaaaacaattacagataagaaaataccatgtaagtttccatgagcagaaatacgtccgcccattttttcaacaattcctaataattgattgtaaaacctgttttgctgatcataagtaatcatactaaacacccattattataaggatccataggccctactacaaggaatgcaaaaatgaataataaaattattaaaattattccttttgcaaagattatttcaacttcatacttttctctccaggacacttttttatgaagtctaactggagtaggtttattgttgaagagactcataattgagcctccaacactttatacttaacttcttttttcataccaggagtattaaggaaatctttcaattcccttaatctggtttgttcttcaacatactctgcatactccccgtataatgtttcgaacaattctctttgttcaggatattcctgtgctagaaatacgaacagttcatctatttcacagtaatctttgtactgtgactggtttatacgatataacgcattaccaatcttgttttcatttttccaaaattcaggagtgaacattcatttacccccgtaagctagttcacctgcacgagcaaactccaacatttgatcatcagacaattcatagatttgaaattgcttatattcatcagccaacctatgatctatttcttcctgtgtttcataatcctcagacaataatatctgttcaagataatcaaactcgacctcatcttcaggtggagtaatcgtatacattgagttgttgtatctgcaactcatacgcccacactcctgaatttttggagttcttcttcatagactctgttttgagcctgaagcctttttgcttttaaaaatacatgttcaatttcttctttggaagttaaccttccaaaaacttcaatcttattattatccatattaccaactcgtggattttttagaagatcttcttcagagttgccgctctgattttttcttcttcaaataactattatacttaatactatataaaggttattatatttacctttattaaaggtaaatatatatactgcaaaacagaatatattatataagaaatataaaaatggaggtatgcctattttagaatatacaacaaaattaagtaaaggcggacctaattctattaggtcaatagtcccccaagatgtgataaaattattagaattagaactgggggattcattacattggattgtaaatattgatgaaggaataactgtatctattgaaaaagctgaaaaataatcagttaatccttcacaaatatttttagtaaatattaaatatctttttttataataaatagtattggttactaaaatttttatcaactcgtggtatctattttttgtagcctctagtttttgagaggcgagatgttgccgcatcacagaccccctcatcaactaggaattacatttacttattttgcatttaattgttctttttcaacctttttttgttctgataacatcataaaatatgtaaagtataatctatttacaaaaccatattccccactattttctctcccaatagactctataatcccaaggtctttagctctctttaaaaaatcattaaaagctctcttctccccatttgtcaaaatatttttcacatctgattttttaaaatccattaatctattttcccctaatttaagaaaaatatcaatatatgtatcgcttctaattttatttaatttggattttaattgttttttagctaattcatctgttgcatcagtaataccagaaaatactatagattcatttatttttaaattatcttgagcattccaaaaaattgaatcaccaatttgttgcattattaaaggcataccccatgaaaaataaaccataggttctatagaattatatccatcttcaaactcaatgcctacattattaaatgaagttgtgaagaaatcttcaatatcattatcctctaaattatcaatttcaattaaattaaacattctagaaaatgattcattaattgaacataatttttcaaattcatgagggtaacttattaatgtgaaaacaacaggcatatgatattcattaaccaataatgtttcagataatcctttataccaatctgtaaactcttcattatcagataaaccattaagatcatcaacaacaataaatattccataatcttcaggtaattctttacaagtagtaatcaaaaaatctgcaaaatgttttttaatattatgaattaaattagtatcattatcttttaaagatactccagtccctgcaactttaaattcattaatattattcaaaattttatcaattacacttttacctaaataatcctttttaaattcttttaataatccatcaagtaacttttgtattaattcatcaatagtttttccaccaccattgttaatatggattggaatcatttgaaaatcatcttctgcttttcttgaaacataatttataaatgatgttttacccattcctctctttccagttatgaaaaaatgttcgggagctccttgatttttaactttaggtaaatattttataattttgttgatatcttcttttcttcctttaaaattatctggagatactggtctccctggttcaaaaggactttctgggaaatcttgtgacatgatatttttacccctcaatactcatttattacaatttacattttgtatttaactgtcttatcaaatttttattactgtatcagctagaaaatctggtgtaacattccgtttctttttttgatttaaaatagtattggaccatttactgaactgtgtagcttctttactattgacgcgatagcctacggaaatcatagcatctaaattatagagtatgggttgagtatttgcttcaggattaattttaataacgattccactaatagtggaatcgtttggattaacagttacttcatctttaacaagttctccactatcaaagatattatttaaatgtttactaatgttttttggtgttttattaaataaattagccattgttttttgtgttgcccataatgtttcgttttcttgatcaatgataactttaattgatactggcccatcttcacttttatataaaaatgtttcaataagattactttccaccatgatttatataacctctacaattttactcttcaaaaaagtttttttattcttgtttttatatttatgttataagttaatataaatgtttctattcaaaaaataaaccttaaaaatacagtatattcaatgaaaataatgaaacactaaacattattcagtatgaaaaaatgaaacgcattgtgttctaaattttggtgtttaattagttttgtgaactgttaggttattgttcatttttataatgaatattttttatctttttttgttattaaaatgtattgatatttttaattaaaaaaatagaaacctttatatactatgtagtacaatataataatagaaatacatggaggtgaaaagttgtcagaatcagaggcaataaaaatcataatattgtctttgagtataatcctagaaatactcaaaagacaaaaacacaagtaggaggttaaccccctcttacaattattatattttttattgcttcttatataaatttttcttaaaaaggaggtgaaaaacaaaatgacaataacaagcatcataatactaatattattaatcatattagcaatattaatctacattaatagaaaccaaaaatatacttggataagtttaataatatggttggttattttaataagtttcatctacgaaacattaatctaacccccccatattttttttatattataagccaaaaaaaaggaagatgataaaaaatgactgactacaccagcataagaataaaaaaagaaatagctgaaaaaatacaattaataaaaatacaaaacaactgcaaatcactaaacgaaacactagaacaactaataccccgaacagtaaacgaaaactacgagttcataaaagaacaaccaatattcacaataaacaacaaacccataacattcacagacctgaaaaacaacaatacaggcaaaacatggggaaatgaaaaacaaaacgcaacaatagtatttaaagataaacaaggagcatttataagattcaatgatgaagatgaagtattcttagaatactaccacttcatctaaaaccattctttttttaatattaaaaataaatccataaaacctcaaatttttaaagttttaaaaatttgttccataaggtacatagcatcatccaattcaccttcagagatattataaaaaggatcataatctgctttttttcttaaattaaataaatcatatagtttattcctaacagaatactgattgacctcaagaagttcatattgaacttgcccatgttctgtaataaactcattttcaccaaagtcttctttagacaatggtttaaatttataaacttcctgaagccacaaactaacatataaataagcactgtaatagcacctattgataatagtactaaacacacaacgattatcgcaattttcagggattaattcttctttattttcatataactgcttagtgaatttatataattcatattgagggtatttttcaatattaatgtataaaacccccaataaattatttaattaaaatataggtatctttatagaaatcaaacatattattttcctttgaaaagataaccatatcattaataatttggtctaacagagaatatttttctttcctatcaatatgaaatggaacattaattgtaaatactttttcaggatatgatccatcatacactgtatcaatagatatattttttcctaatcctaaattgtcagaaatttttataagttcatcataaaattcagtgattttactttcaaaagactctgtggatgattttttaaaaaacttatttttaaaaaaatcccctacaatattagaaggtttattatatccataatttttatcaactgaatacaccatagacaccacctataaatgtttattctaaagtttttctaataatatccaatgtttcaggattattttgaacatcaataagaactgtattactttttatttcttcatcattcctataagataaagtaagaatatccttatttatttccccatcttttaaaacaaaagctgcatttataggtataacttcaagattttcaaagttttcttcaagtttttttaaaaaatcaacattaaaaacttctttaagggaaatcccctcttgaagcactcttaagcttcttgcatcctttttcatttaacttcctccaaaatagttatttaataaatatcttttattaaacatataaaccttgtcaaaaaatttttaaattaataaatcttcaaagtcattttctttgaaattttcttcttttttctcaatatttcgtaatctttcatcaatatcatagattaatttttcataatctttgattttttcatctttttcaacaattgtattttctaattttaaaaattctggagatttaacagtgatcttttcaacttctttactaattgataatgctggtaaatgttgaatatattcctgttttaaatcagcaggattggtcatgaaatatacttcatcagtactgttttttgatttgccttgcaggtcatttactttgtccagactcataccatcattatatagtgttgaggcgtggaattttctgagcatgtggctgcggaatctgttgtagtttcctgcttttcctaatcctaattcattgtttattttaatgaattgttggtttaggtagtcttcatgtattttgaatagttgggattctggtgttaaattttgtcttgatagtaagtgatgatttatagctgtgactgcttctggactgcaataggtaatataatatttctgtgttttttgccttaaaatattaaaagtagggacaacattatcaatattatttaacacatctatcatttccataatattatctgtattatggtattctttagttgcgttcatatagtccattacagttaaatttaatgtttctcttctggcacaacctgaactactcatgaataatataatagctttcatggtagaagtacatatattaactgcttcacgaataatttctttatcaggtaaatctttaaaactgatcggtttgggattattataactttttttatcaattcgtggcagatcatgtatttcaatttcaaaatatttatatacaacaagaactggtgtgaaagtatttgatacagtgttataataataattatccatcaaatacttgcgaaaatttaataatcttctttttaatgtacgatgtttccatcgtataccctgttcttcttctttttctgcttcttcgattaattcggctaatgataacttattcaattcacaatatttatttattgaatgtttgtaaatatgctgtgttgtttttttatgatttttaacagaatggatctcatttaggatttcttcattggttctcatatttatcaaatctatttttctctttttatattgtttgatgtagagtgcatttgataagtatgtgataatatgtctactgtcgtgtctacaacgagttaattataacatacttaaaacattctaatctattaaaatattatatcaaaaataggttttaaattttatcccccaaaattaaacccttatcctcgtactgcgtttataaaaaatggaagtaattaagataaatttctccgtattcatcttcaaatctgattaatgctccaaggttatctttatagattaatgttgctttttccccattactccattgtgtgcctacttccgattgttttaattcattccatgatacatttaaaactgtttttttgtttattagtgtgaatgctggttgttcttgtatgtattcttcaggtgttgatgttccatcaggtaataaatgttttactgtttcattaactgatttgtatccgttgatttcctgtatttcttttatcttgttaattaaatttacttcaagttttatagtacttctttccattatttaattcctcctttaaaaaaataataaaaaaattatagtctttatattaaatggattataatgatgactataatatttattataagcattattgcgtttcgtgtcctgttttcttcatttttcattgaatataataatactactgcaagtattattaaaattatttgaattaatgttaatactgtggttatcataattccccatcccttttatttaaatattagtaatgacatataatttattataaattattttaaagagggattaattttaatcccccattagggataatattattgatattatccccataaccatatttattagaaaatatatgtcatttttatcaatgtttttcacctccttgtagttctattattatattggttactatactatataaaggtttctattttttactacttatttttataaaaaaataaaagaattgttgataaaacaacaatcttattgatacaaatctgagaaaatccaagctggatcatacgctaaaatcgtaccattcatacaccaattacttgaaacactgtttccatctaaaactgctgcagggtcacgataaatccaagaacccccagtatacttagaatgtttcaatctgagccttacatgcccagtcccacttctacatcgtacatgaatgaattgtacattataacctaatgcaagtcctaaacggtaaaataattgacttgaatcagtacaattaactccttgcttattatatatcttattaatagtttcctgagtattatatttggagttataataatatccatatcctcttcctcggattttgttcaagcaagagtctatatctgttacaccaccaaatttgtcggtgaacctttttaatgtgatgtctgtggtgtcgctggttgttccgttgccatgtgggtcttcaagataaacaatagctggactaacaccatttaatacttcataagctgatacacgattagccatactaacataacaattcttataatactttgttccatcagtatcagacacataatttgggaggaaacttaaatctttattatcaaatattgcacattcaacaaactctgcaataagactgtcttgttgataattcgtacctctctctttaattgcttttataattgtgccataatcagctacacgattcatgtaactatacacttgtcttgtattaaactttttattattattcactaaccaataagcaattctattcatcaaataagtgtattttgtaaaactaatatgttcattaaccatttattttcatctcctttttaaattttaaatcatctaaatattcctttaaaatactgcattttattattgcatcttcaaaacaacaaggaacctctttatcacaaggatcataaaatgcacatttttcacattcttcactaatcattccaaaataatcctcctatcaatttttaatgtacaatcagtaatcttgtttgttctaaacgaggattctctccagcagctcctctttttacaataccttccaacatataacttccagggtctaatctaatcggcagtattgctctaccattagcatcagttgttctttcataagaaacaccatttacaataaaattaattaccacattttcccctgtgttaatttcaggattggaagaaataattttaccgtaaacagaatttccataatttaattctagtttaactccatcttctccagcaaggtctgataagcgacccattactaatctagtgttttttaaatcatccattgtggtttctaatatttcttgtcttgcaaatgtttcagaatgttcatcatccaccaaatctgcatttaaacctgaacctgctccatccacttcttttaatttatttaaaatagtggtgggtgtttcaatgtttccatctttaccatcttttcctgcaggtccagtatcacctttacttcctttatcacctttaggacctgttggtcctgttggtccagtattgcctttagggcctttgaaattccccagtaatatttttttaaccattaaaaaaaacctcttcatttatttaattatactcctattttcattttcacaccagacacattataactcatacttgtaccgctactccatgcacctgcttttgtaattaatctccacctattaggattaatttctgctcctcgactcttattggctgtgttaattgtttgagaatcatcacataatacataacttaacttatcatgtcttactttgaaaattaaaccaaaatattttcctgcaacattcccatatgtgcccttttggtcgtaagttctatttgttccaccatcacaaactatatcaacccatgtacggccttgctgaggggtgcatactacacttattttttcgtttgtttctgggttcaataagataaattgtatattagagttagttcctaagctgttaggactccatgataaaaataagtcaaagttttcaagattaaaagtatctgtacgatacccttcttgagtgctctgcttgtagggtatttgtatggtatcgtgaacatgtgagtgagtaatacttttatctgcaatactgcttccttcaggcaatataacatcagataaaaagaaactaaaatctcttgtattaatattggttttaccatccaagcttgattgaagatcagtaatattgctgatactgtgactgtgactattgttagctttaccgttcaacgcattagtcacgattttattctgtacaggattaatagaagaactacttaatgatgaatcaacaactgttttattagcaccatcatcaatattttccaattttttgtcatgatcataaacaaaaccatttacacttgactgtctattaattccatattcatacacccacatgccatcatcactatcctgatcaaggtctatactatcacttgtatggctgtggttaacatttgctttatttgctaattgagtattcatttctgtttgtgtaacataccctgataagtcaactgttgtttcagttgaaccaattttttcaaatttattattaacccaaatatactcatcataaatattaccctcaacagaagctgaagtatttaattttaaatacattactccaacttcccctgtagatggtaatgaatttactattgttgctgtaaaaccaattaatgaatttatctgagaattaatattatttactttcgcatctatttcagtttcagtataatacctttcatcatgattatgtccaacatttgattttccacctaattttgaatcaatttctgttttcttataataatcatttaaatgtttaacagcaatataattgccaacatgtacctgataactatttgctccatcttcactagctatatttaaatataaatcaccagtagtttcatcatgttcaaatattggagggttggtttcatcattacaataacaccatagatttccttcagcatctcctgctaaagtgaaaaaacctggtggaggtactgtaatatttaataatgggtttgcccatttgtcgaacaattcatcaatggctttacttactgtattggcatctgtctttaatatgtcgatgtctaggaaatcttcaatgtttcctatttttgtgcctaggtttgtttctcttaatcttgcacatgcattatttaatgcttttctttcattttcagttagtttagtcatattacactctctcgtttaacattacctattttaatctctttgtcaccatttagataaggattataacttatttctgttatttgtgctttgaacacctctgaatctgggaatcttacatatac